SEQUENCE LISTING (94 sequences)

SEQ ID NO: 1 - protein, 223 aa, Acremonium cellulolyticus
MKLSLAAIGICTTAAVAFPSGLTQHATGDLSKRQSITTSQTGTNNGYYYSFWTNGGGEVTYTNGDNGEYSVTWVNCGDFTSGKGWNPANAQTVTYSGEFNTSGNAYLAVYGWTTDPLVEYYILESYGTYNPSSGLTLLGQVTSDGGTYDIYSTQRVDQPSIEGTSTFNQYWSVRTEKRVGGTVTTANHFAAWKALGLEMGTYNYMIVSTEGYESSGSSTITVS

SEQ ID NO: 2 - protein, 189 aa, Acremonium cellulolyticus
SITTSQTGTNNGYYYSFWTNGGGEVTYTNGDNGEYSVTWVNCGDFTSGKGWNPANAQTVTYSGEFNTSGNAYLAVYGWTTDPLVEYYILESYGTYNPSSGLTLLGQVTSDGGTYDIYSTQRVDQPSIEGTSTFNQYWSVRTEKRVGGTVTTANHFAAWKALGLEMGTYNYMIVSTEGYESSGSSTITVS

SEQ ID NO: 3 - protein, 189 aa, Acremonium cellulolyticus
SITTSQTGTNNGYYYSFWTNGGGEVTYTNGDNGEYSVTWVNCGAFTSGKGWNPANAQTVTYSGEFNTSGNAYLAVYGWTTDPLVEYYILESYGTYNPSSGLTLLGQVTSDGGTYDIYSTQRVDQPSIEGTSTFNQYWSVRTEKRVGGTVTTANHFAAWKALGLEMGTYNYMIVSTEGYESSGSSTITVS

SEQ ID NO: 4 - protein, 189 aa, Acremonium cellulolyticus
SITTSQTGTNNGYYYSFWTNGGGEVTYTNGDNGEYSVTWVNCGDFASGKGWNPANAQTVTYSGEFNTSGNAYLAVYGWTTDPLVEYYILESYGTYNPSSGLTLLGQVTSDGGTYDIYSTQRVDQPSIEGTSTFNQYWSVRTEKRVGGTVTTANHFAAWKALGLEMGTYNYMIVSTEGYESSGSSTITVS

SEQ ID NO: 5 - protein, 189 aa, Acremonium cellulolyticus
SITTSQTGTNNGYYYSFWTNGGGEVTYTNGDNGEYSVTWVNCGDFTSGKGWNPANAQTVTYSGEFNTSGNAYLAVYGWTTDPNVEYYILESYGTYNPSSGLTLLGQVTSDGGTYDIYSTQRVDQPSIEGTSTFNQYWSVRTEKRVGGTVTTANHFAAWKALGLEMGTYNYMIVSTEGYESSGSSTITVS

SEQ ID NO: 6 - protein, 189 aa, Acremonium cellulolyticus
SITTSQTGTNNGYYYSFWTNGGGEVTYTNGDNGEYSVTWVNCGDFTSGKGWNPANAQTVTYSGEFNTSGNAYLAVYGWTTDPLVEYYILESYGTYNPSSGLTLLGQVTSDGGTYDIYSTQAVDQPSIEGTSTFNQYWSVRTEKRVGGTVTTANHFAAWKALGLEMGTYNYMIVSTEGYESSGSSTITVS

SEQ ID NO: 7 - protein, 189 aa, Acremonium cellulolyticus
SITTSQTGTNNGYYYSFWTNGGGEVTYTNGDNGEYSVTWVNCGDFTSGKGWNPANAQTVTYSGEFNTSGNAYLAVYGWTTDPLVEYYILESYGTYNPSSGLTLLGQVTSDGGTYDIYSTQRVDQPSIEGTSTFNAYWSVRTEKRVGGTVTTANHFAAWKALGLEMGTYNYMIVSTEGYESSGSSTITVS

SEQ ID NO: 8 - protein, 189 aa, Acremonium cellulolyticus
SITTSQTGTNNGYYYSFWTNGGGEVTYTNGDNGEYSVTWVNCGDFTSGKGWNPANAQTVTYSGEFNTSGNAYLAVYGWTTDPLVEYYILESYGTYNPSSGLTLLGQVTSDGGTYDIYSTQRVDQPSIEGTSTFNQYWSVRTEKRVGGTVTTANHFAAWKALGLEMGTYWYMIVSTEGYESSGSSTITVS

SEQ ID NO: 9 - protein, 189 aa, Acremonium cellulolyticus
SITTSQTGTNNGYYYSFWTNGGGEVTYTNGDNGEYSVTWVNCGAFTSGKGWNPANAQTVTYSGEFNTSGNAYLAVYGWTTDPLVEYYILESYGTYNPSSGLTLLGQVTSDGGTYDIYSTQAVDQPSIEGTSTFNQYWSVRTEKRVGGTVTTANHFAAWKALGLEMGTYNYMIVSTEGYESSGSSTITVS

SEQ ID NO: 10 - protein, 189 aa, Acremonium cellulolyticus
SITTSQTGTNNGYAYSFWTNGGGEVTYTNGDNGEYSVTWVNCGDFTSGKGWNPANAQTVTYSGEFNTSGNAYLAVYGWTTDPLVEYYILESYGTYNPSSGLTLLGQVTSDGGTYDIYSTQRVDQPSIEGTSTFNQYWSVRTEKRVGGTVTTANHFAAWKALGLEMGTYNYMIVSTEGYESSGSSTITVS

SEQ ID NO: 11 - protein, 189 aa, Acremonium cellulolyticus
SITTSQTGTNNGYYYSFWTNGGGEVTYTNGDNGEYSVTWVNCGDFTSGKGWNPANAQTVTYSGEFNTSGNAYLAVYGATTDPLVEYYILESYGTYNPSSGLTLLGQVTSDGGTYDIYSTQRVDQPSIEGTSTFNQYWSVRTEKRVGGTVTTANHFAAWKALGLEMGTYNYMIVSTEGYESSGSSTITVS

SEQ ID NO: 12 - protein, 189 aa, Acremonium cellulolyticus
SITTSQTGTNNGYYYSFWTNGGGEVTYTNGDNGEYSVTWVNCGDFTSGKGWNPANAQTVTYSGEFNTSGNAYLAVYGWTTDPLVEYAILESYGTYNPSSGLTLLGQVTSDGGTYDIYSTQRVDQPSIEGTSTFNQYWSVRTEKRVGGTVTTANHFAAWKALGLEMGTYNYMIVSTEGYESSGSSTITVS

SEQ ID NO: 13 - protein, 189 aa, Acremonium cellulolyticus
SITTSQTGTNNGYYYSFWTNGGGEVTYTNGDNGEYSVTWVNCGDFTSGKGWNPANAQTVTYSGEFNTSGNAYLAVYGWTTDPLVEYYIYESYGTYNPSSGLTLLGQVTSDGGTYDIYSTQRVDQPSIEGTSTFNQYWSVRTEKRVGGTVTTANHFAAWKALGLEMGTYNYMIVSTEGYESSGSSTITVS

SEQ ID NO: 14 - protein, 189 aa, Acremonium cellulolyticus
SITTSQTGTNNGYYYSFWTNGGGEVTYTNGDNGEYSVTWVNCGDFTSGKGWNPANAQTVTYSGEFNTSGNAYLAVYGWTTDPLVEYYILESYGYYNPSSGLTLLGQVTSDGGTYDIYSTQRVDQPSIEGTSTFNQYWSVRTEKRVGGTVTTANHFAAWKALGLEMGTYNYMIVSTEGYESSGSSTITVS

SEQ ID NO: 15 - protein, 189 aa, Acremonium cellulolyticus
SITTSQTGTNNGYYYSFWTNGGGEVTYTNGDNGEYSVTWVNCGDFTSGKGWNPANAQTVTYSGEFNTSGNAYLAVYGWTTDPLVEYYILESYGTYNPSSGLTLLGQVTSDGGTYDIYSTQRVDQPSIEGTSTANQYWSVRTEKRVGGTVTTANHFAAWKALGLEMGTYNYMIVSTEGYESSGSSTITVS

SEQ ID NO: 16 - protein, 189 aa, Acremonium cellulolyticus
SITTSQTGTNNGYYYSFWTNGGGEVTYTNGDNGEYSVTWVNCGDFTSGKGWNPANAQTVTYSGEFNTSGNAYLAVYGWTTDPLVEYYILESYGTYNPSSGLTLLGQVTSDGGTYDIYSTQRVDQPSIEGTSTFNQYASVRTEKRVGGTVTTANHFAAWKALGLEMGTYNYMIVSTEGYESSGSSTITVS

SEQ ID NO: 17 - protein, 189 aa, Acremonium cellulolyticus
SITTSQTGTNNGYYYSFWTNGGGEVTYTNGDNGEYSVTWVNCGDFTSGKGWNPANAQTVTYSGEFNTSGNAYLAVYGWTTDPLVEYYILESYGTYNPSSGLTLLGQVTSDGGTYDIYSTQRVDQPSIEGTSTFNQYWSVRTEKRVGGTVTTANHFAAWKALGLEMGTYNYMIVSTEGAESSGSSTITVS

SEQ ID NO: 18 - protein, 189 aa, Acremonium cellulolyticus
CITTSQTGTHNGYYYSFWTNGGGEVTMCLGPGGEYSVTWVNCGAFTSGKGWNPANAQTVTYSGEFNPNGNAYLAVYGWTTDPLVEYYILESYGTYNPSSGLTLLGQVTSDGGTYDIYSTQRVDQPSIEGTSTFNQYWSVRTEKRVGGTVTTANHFAAWKALGLEMGTYNYMIVSTEGYESSGSSTITVS

SEQ ID NO: 19 - protein, 189 aa, Acremonium cellulolyticus
CITTSQTGTHNGYYYSFWTNGGGEVTMCLGPGGEYSVTWVNCGDFASGKGWNPANAQTVTYSGEFNPNGNAYLAVYGWTTDPLVEYYILESYGTYNPSSGLTLLGQVTSDGGTYDIYSTQRVDQPSIEGTSTFNQYWSVRTEKRVGGTVTTANHFAAWKALGLEMGTYNYMIVSTEGYESSGSSTITVS

SEQ ID NO: 20 - protein, 189 aa, Acremonium cellulolyticus
CITTSQTGTHNGYYYSFWTNGGGEVTMCLGPGGEYSVTWVNCGAFTSGKGWNPANAQTVTYSGEFNPNGNAYLAVYGWTTDPLVEYYILESYGTYNPSSGLTLLGQVTSDGGTYDIYSTQAVDQPSIEGTSTFNQYWSVRTEKRVGGTVTTANHFAAWKALGLEMGTYNYMIVSTEGYESSGSSTITVS

SEQ ID NO: 21 - DNA, 29 bp, Artificial
  Forward primer for a mutation
ttgcggtgcgtttacatctggcaagggct

SEQ ID NO: 22 - DNA, 28 bp, Artificial
  Reverse primer for mutation
gatgtaaacgcaccgcaattgacccagg

SEQ ID NO: 23 - DNA, 30 bp, Artificial
  Forward primer for a mutation
gtgactttgcgtctggcaagggctggaatc

SEQ ID NO: 24 - DNA, 29 bp, Artificial
  Reverse primer for mutation
ttgccagacgcaaagtcaccgcaattgac

SEQ ID NO: 25 - DNA, 29 bp, Artificial
  Forward primer for a mutation
gatcctaacgtcgaatactacatcctgga

SEQ ID NO: 26 - DNA, 28 bp, Artificial
  Reverse primer for mutation
ttcgacgttaggatcagttgtccaaccg

SEQ ID NO: 27 - DNA, 29 bp, Artificial
  Forward primer for a mutation
caacacaggcggtcgaccaaccctccatc

SEQ ID NO: 28 - DNA, 29 bp, Artificial
  Reverse primer for mutation
ggtcgaccgcctgtgttgagtagatatcg

SEQ ID NO: 29 - DNA, 27 bp, Artificial
  Forward primer for a mutation
cttcaatgcgtactggtcggttcgcac

SEQ ID NO: 30 - DNA, 28 bp, Artificial
  Reverse primer for mutation
accagtacgcattgaaggtggaagttcc

SEQ ID NO: 31 - DNA, 29 bp, Artificial
  Forward primer for a mutation
gtacttattggtatatgattgtgtctaca

SEQ ID NO: 32 - DNA, 29 bp, Artificial
  Reverse primer for mutation
atcatataccaataagtacccatttcaag

SEQ ID NO: 33 - DNA, 28 bp, Artificial
  Forward primer for a mutation
ctactacgcgtcgttctggaccaacggc

SEQ ID NO: 34 - DNA, 28 bp, Artificial
  Reverse primer for mutation
cagaacgacgcgtagtagccgttgtgcg

SEQ ID NO: 35 - DNA, 30 bp, Artificial
  Forward primer for a mutation
gtttacggtgcgacaactgatcctcttgtc

SEQ ID NO: 36 - DNA, 30 bp, Artificial
  Reverse primer for mutation
cagttgtcgcaccgtaaacggcgagataag

SEQ ID NO: 37 - DNA, 29 bp, Artificial
  Forward primer for a mutation
cgaatacgcgatcctggagtcctacggta

SEQ ID NO: 38 - DNA, 29 bp, Artificial
  Reverse primer for mutation
ctccaggatcgcgtattcgacaagaggat

SEQ ID NO: 39 - DNA, 30 bp, Artificial
  Forward primer for a mutation
atactacatctatgagtcctacggtacaat

SEQ ID NO: 40 - DNA, 30 bp, Artificial
  Reverse primer for mutation
gtaggactcatagatgtagtattcgacaag

SEQ ID NO: 41 - DNA, 28 bp, Artificial
  Forward primer for a mutation
ctacggttactataacccatcatctggc

SEQ ID NO: 42 - DNA, 28 bp, Artificial
  Reverse primer for mutation
gggttatagtaaccgtaggactccagga

SEQ ID NO: 43 - DNA, 29 bp, Artificial
  Forward primer for a mutation
cttccaccgcgaatcagtactggtcggtt

SEQ ID NO: 44 - DNA, 28 bp, Artificial
  Reverse primer for mutation
gtactgattcgcggtggaagttccctcg

SEQ ID NO: 45 - DNA, 29 bp, Artificial
  Forward primer for a mutation
atcagtacgcgtcggttcgcacagagaag

SEQ ID NO: 46 - DNA, 29 bp, Artificial
  Reverse primer for mutation
cgaaccgacgcgtactgattgaaggtgga

SEQ ID NO: 47 - DNA, 29 bp, Artificial
  Forward primer for a mutation
cagaaggcgcggagagcagtggttctagt

SEQ ID NO: 48 - DNA, 29 bp, Artificial
  Reverse primer for mutation
tgctctccgcgccttctgtagacacaatc

SEQ ID NO: 49 - DNA, 27 bp, Artificial
  Forward primer for a mutation
gatatacatatgtgtatcacgacgagc

SEQ ID NO: 50 - DNA, 27 bp, Artificial
  Reverse primer for mutation
gctcgtcgtgatacacatatgtatatc

SEQ ID NO: 51 - DNA, 30 bp, Artificial
  Forward primer for a mutation
actgggacgcacaacggctactactactcg

SEQ ID NO: 52 - DNA, 29 bp, Artificial
  Reverse primer for a mutation
cgagtagtagtagccgttgtgcgtcccag

SEQ ID NO: 53 - DNA, 28 bp, Artificial
  Forward primer for a mutation
gtcacctactgtaatggtgacaatggcg

SEQ ID NO: 54 - DNA, 30 bp, Artificial
  Reverse primer for a mutation
ttcgccattgtcaccattacagtaggtgac

SEQ ID NO: 55 - DNA, 30 bp, Artificial
  Forward primer for a mutation
acctacacattaggtgacaatggcgaatac

SEQ ID NO: 56 - DNA, 29 bp, Artificial
  Reverse primer for a mutation
gtattcgccattgtcacctaatgtgtagg

SEQ ID NO: 57 - DNA, 27 bp, Artificial
  Forward primer for a mutation
ggagaatttaatcccagcggaaacgct

SEQ ID NO: 58 - DNA, 27 bp, Artificial
  Reverse primer for a mutation
agcgtttccgctgggattaaattctcc

SEQ ID NO: 59 - DNA, 27 bp, Artificial
  Forward primer for a mutation
gaatttaatacaaacggaaacgcttat

SEQ ID NO: 60 - DNA, 27 bp, Artificial
  Reverse primer for a mutation
ataagcgtttccgtttgtattaaattc

SEQ ID NO: 61 - DNA, 30 bp, Artificial
  Forward primer for a mutation
gaagtcaccatgacaaatggtgacaatggc

SEQ ID NO: 62 - DNA, 30 bp, Artificial
  Reverse primer for a mutation
gccattgtcaccatttgtcatggtgacttc

SEQ ID NO: 63 - DNA, 29 bp, Artificial
  Forward primer for a mutation
cctacacaaatggtcctaacggcgaatac

SEQ ID NO: 64 - DNA, 29 bp, Artificial
  Reverse primer for a mutation
gtattcgccgttaggaccatttgtgtagg

SEQ ID NO: 65 - DNA, 27 bp, Artificial
  Forward primer for a mutation
acaaatggtgatggaggcgaatacagc

SEQ ID NO: 66 - DNA, 27 bp, Artificial
  Reverse primer for a mutation
gctgtattcgcctccatcaccatttgt

SEQ ID NO: 67 - DNA, 735 bp, Acremonium cellulolyticus
atgaagctctctctggctgcaattggcatttgcacaactgccgccgtcgcctttccatctggacttactcaacacgctacgggagatctcagcaagcgtcaatcaatcacgacgagccagactgggacgaacaacggctactactactcgttctggaccaacggcggaggagaagtcacctacacaaatggtgacaatggcgaatacagcgtgacctgggtcaattgcggtgactttacatctggcaagggctggaatccagctaatgcacagtaagttttctattttgttgtgttctaagcttatattttacatactcacatcggaatttgaaggactgtcacctactctggagaatttaatacctctggaaacgcttatctcgccgtttacggttggacaactgatcctcttgtcgaatactacatcctggagtcctacggtacatataacccatcatctggccttacattacttggccaggttactagcgatggtggtacgtacgatatctactcaacacagcgtgtcgaccaaccctccatcgagggaacttccaccttcaatcagtactggtcggttcgcacagagaagcgagtcggcggaactgtcaccacggccaaccactttgcagcatggaaggcacttggacttgaaatgggtacttataactatatgattgtgtctacagaaggctacgagagcagtggttctagtaccatcaccgtgtcctag

SEQ ID NO: 68 - DNA, 570 bp, Acremonium cellulolyticus
tcaatcacgacgagccagactgggacgaacaacggctactactactcgttctggaccaacggcggaggagaagtcacctacacaaatggtgacaatggcgaatacagcgtgacctgggtcaattgcggtgactttacatctggcaagggctggaatccagctaatgcacagactgtcacctactctggagaatttaatacctctggaaacgcttatctcgccgtttacggttggacaactgatcctcttgtcgaatactacatcctggagtcctacggtacatataacccatcatctggccttacattacttggccaggttactagcgatggtggtacgtacgatatctactcaacacagcgtgtcgaccaaccctccatcgagggaacttccaccttcaatcagtactggtcggttcgcacagagaagcgagtcggcggaactgtcaccacggccaaccactttgcagcatggaaggcacttggacttgaaatgggtacttataactatatgattgtgtctacagaaggctacgagagcagtggttctagtaccatcaccgtgtcctag

SEQ ID NO: 69 - DNA, 570 bp, Acremonium cellulolyticus
tcaatcacgacgagccagactgggacgaacaacggctactactactcgttctggaccaacggcggaggagaagtcacctacacaaatggtgacaatggcgaatacagcgtgacctgggtcaattgcggtgcgtttacatctggcaagggctggaatccagctaatgcacagactgtcacctactctggagaatttaatacctctggaaacgcttatctcgccgtttacggttggacaactgatcctcttgtcgaatactacatcctggagtcctacggtacatataacccatcatctggccttacattacttggccaggttactagcgatggtggtacgtacgatatctactcaacacagcgtgtcgaccaaccctccatcgagggaacttccaccttcaatcagtactggtcggttcgcacagagaagcgagtcggcggaactgtcaccacggccaaccactttgcagcatggaaggcacttggacttgaaatgggtacttataactatatgattgtgtctacagaaggctacgagagcagtggttctagtaccatcaccgtgtcctag

SEQ ID NO: 70 - DNA, 570 bp, Acremonum cellulolyticus
tcaatcacgacgagccagactgggacgaacaacggctactactactcgttctggaccaacggcggaggagaagtcacctacacaaatggtgacaatggcgaatacagcgtgacctgggtcaattgcggtgactttgcgtctggcaagggctggaatccagctaatgcacagactgtcacctactctggagaatttaatacctctggaaacgcttatctcgccgtttacggttggacaactgatcctcttgtcgaatactacatcctggagtcctacggtacatataacccatcatctggccttacattacttggccaggttactagcgatggtggtacgtacgatatctactcaacacagcgtgtcgaccaaccctccatcgagggaacttccaccttcaatcagtactggtcggttcgcacagagaagcgagtcggcggaactgtcaccacggccaaccactttgcagcatggaaggcacttggacttgaaatgggtacttataactatatgattgtgtctacagaaggctacgagagcagtggttctagtaccatcaccgtgtcctag

SEQ ID NO: 71 - DNA, 570 bp, Acremonium cellulolyticus
tcaatcacgacgagccagactgggacgaacaacggctactactactcgttctggaccaacggcggaggagaagtcacctacacaaatggtgacaatggcgaatacagcgtgacctgggtcaattgcggtgactttacatctggcaagggctggaatccagctaatgcacagactgtcacctactctggagaatttaatacctctggaaacgcttatctcgccgtttacggttggacaactgatcctaacgtcgaatactacatcctggagtcctacggtacatataacccatcatctggccttacattacttggccaggttactagcgatggtggtacgtacgatatctactcaacacagcgtgtcgaccaaccctccatcgagggaacttccaccttcaatcagtactggtcggttcgcacagagaagcgagtcggcggaactgtcaccacggccaaccactttgcagcatggaaggcacttggacttgaaatgggtacttataactatatgattgtgtctacagaaggctacgagagcagtggttctagtaccatcaccgtgtcctag

SEQ ID NO: 72 - DNA, 570 bp, Acremonium cellulolyticus
tcaatcacgacgagccagactgggacgaacaacggctactactactcgttctggaccaacggcggaggagaagtcacctacacaaatggtgacaatggcgaatacagcgtgacctgggtcaattgcggtgactttacatctggcaagggctggaatccagctaatgcacagactgtcacctactctggagaatttaatacctctggaaacgcttatctcgccgtttacggttggacaactgatcctcttgtcgaatactacatcctggagtcctacggtacatataacccatcatctggccttacattacttggccaggttactagcgatggtggtacgtacgatatctactcaacacaggcggtcgaccaaccctccatcgagggaacttccaccttcaatcagtactggtcggttcgcacagagaagcgagtcggcggaactgtcaccacggccaaccactttgcagcatggaaggcacttggacttgaaatgggtacttataactatatgattgtgtctacagaaggctacgagagcagtggttctagtaccatcaccgtgtcctag

SEQ ID NO: 73 - DNA, 570 bp, Acremonium cellulolyticus
tcaatcacgacgagccagactgggacgaacaacggctactactactcgttctggaccaacggcggaggagaagtcacctacacaaatggtgacaatggcgaatacagcgtgacctgggtcaattgcggtgactttacatctggcaagggctggaatccagctaatgcacagactgtcacctactctggagaatttaatacctctggaaacgcttatctcgccgtttacggttggacaactgatcctcttgtcgaatactacatcctggagtcctacggtacatataacccatcatctggccttacattacttggccaggttactagcgatggtggtacgtacgatatctactcaacacagcgtgtcgaccaaccctccatcgagggaacttccaccttcaatgcgtactggtcggttcgcacagagaagcgagtcggcggaactgtcaccacggccaaccactttgcagcatggaaggcacttggacttgaaatgggtacttataactatatgattgtgtctacagaaggctacgagagcagtggttctagtaccatcaccgtgtcctag

SEQ ID NO: 74 - DNA, 570 bp, Acremonium cellulolyticus
tcaatcacgacgagccagactgggacgaacaacggctactactactcgttctggaccaacggcggaggagaagtcacctacacaaatggtgacaatggcgaatacagcgtgacctgggtcaattgcggtgactttacatctggcaagggctggaatccagctaatgcacagactgtcacctactctggagaatttaatacctctggaaacgcttatctcgccgtttacggttggacaactgatcctcttgtcgaatactacatcctggagtcctacggtacatataacccatcatctggccttacattacttggccaggttactagcgatggtggtacgtacgatatctactcaacacagcgtgtcgaccaaccctccatcgagggaacttccaccttcaatcagtactggtcggttcgcacagagaagcgagtcggcggaactgtcaccacggccaaccactttgcagcatggaaggcacttggacttgaaatgggtacttattggtatatgattgtgtctacagaaggctacgagagcagtggttctagtaccatcaccgtgtcctag

SEQ ID NO: 75 - DNA, 570 bp, Acremonium cellulolyticus
tcaatcacgacgagccagactgggacgaacaacggctactactactcgttctggaccaacggcggaggagaagtcacctacacaaatggtgacaatggcgaatacagcgtgacctgggtcaattgcggtgcgtttacatctggcaagggctggaatccagctaatgcacagactgtcacctactctggagaatttaatacctctggaaacgcttatctcgccgtttacggttggacaactgatcctcttgtcgaatactacatcctggagtcctacggtacatataacccatcatctggccttacattacttggccaggttactagcgatggtggtacgtacgatatctactcaacacaggcggtcgaccaaccctccatcgagggaacttccaccttcaatcagtactggtcggttcgcacagagaagcgagtcggcggaactgtcaccacggccaaccactttgcagcatggaaggcacttggacttgaaatgggtacttataactatatgattgtgtctacagaaggctacgagagcagtggttctagtaccatcaccgtgtcctag

SEQ ID NO: 76 - DNA, 570 bp, Acremonium cellulolyticus
tcaatcacgacgagccagactgggacgaacaacggctactacgcgtcgttctggaccaacggcggaggagaagtcacctacacaaatggtgacaatggcgaatacagcgtgacctgggtcaattgcggtgactttacatctggcaagggctggaatccagctaatgcacagactgtcacctactctggagaatttaatacctctggaaacgcttatctcgccgtttacggttggacaactgatcctcttgtcgaatactacatcctggagtcctacggtacatataacccatcatctggccttacattacttggccaggttactagcgatggtggtacgtacgatatctactcaacacagcgtgtcgaccaaccctccatcgagggaacttccaccttcaatcagtactggtcggttcgcacagagaagcgagtcggcggaactgtcaccacggccaaccactttgcagcatggaaggcacttggacttgaaatgggtacttataactatatgattgtgtctacagaaggctacgagagcagtggttctagtaccatcaccgtgtcctag

SEQ ID NO: 77 - DNA, 570 bp, Acremonium cellulolyticus
tcaatcacgacgagccagactgggacgaacaacggctactactactcgttctggaccaacggcggaggagaagtcacctacacaaatggtgacaatggcgaatacagcgtgacctgggtcaattgcggtgactttacatctggcaagggctggaatccagctaatgcacagactgtcacctactctggagaatttaatacctctggaaacgcttatctcgccgtttacggtgcgacaactgatcctcttgtcgaatactacatcctggagtcctacggtacatataacccatcatctggccttacattacttggccaggttactagcgatggtggtacgtacgatatctactcaacacagcgtgtcgaccaaccctccatcgagggaacttccaccttcaatcagtactggtcggttcgcacagagaagcgagtcggcggaactgtcaccacggccaaccactttgcagcatggaaggcacttggacttgaaatgggtacttataactatatgattgtgtctacagaaggctacgagagcagtggttctagtaccatcaccgtgtcctag

SEQ ID NO: 78 - DNA, 570 bp, Acremonium cellulolyticus
tcaatcacgacgagccagactgggacgaacaacggctactactactcgttctggaccaacggcggaggagaagtcacctacacaaatggtgacaatggcgaatacagcgtgacctgggtcaattgcggtgactttacatctggcaagggctggaatccagctaatgcacagactgtcacctactctggagaatttaatacctctggaaacgcttatctcgccgtttacggttggacaactgatcctcttgtcgaatacgcgatcctggagtcctacggtacatataacccatcatctggccttacattacttggccaggttactagcgatggtggtacgtacgatatctactcaacacagcgtgtcgaccaaccctccatcgagggaacttccaccttcaatcagtactggtcggttcgcacagagaagcgagtcggcggaactgtcaccacggccaaccactttgcagcatggaaggcacttggacttgaaatgggtacttataactatatgattgtgtctacagaaggctacgagagcagtggttctagtaccatcaccgtgtcctag

SEQ ID NO: 79 - DNA, 570 bp, Acremonium cellulolyticus
tcaatcacgacgagccagactgggacgaacaacggctactactactcgttctggaccaacggcggaggagaagtcacctacacaaatggtgacaatggcgaatacagcgtgacctgggtcaattgcggtgactttacatctggcaagggctggaatccagctaatgcacagactgtcacctactctggagaatttaatacctctggaaacgcttatctcgccgtttacggttggacaactgatcctcttgtcgaatactacatctatgagtcctacggtacatataacccatcatctggccttacattacttggccaggttactagcgatggtggtacgtacgatatctactcaacacagcgtgtcgaccaaccctccatcgagggaacttccaccttcaatcagtactggtcggttcgcacagagaagcgagtcggcggaactgtcaccacggccaaccactttgcagcatggaaggcacttggacttgaaatgggtacttataactatatgattgtgtctacagaaggctacgagagcagtggttctagtaccatcaccgtgtcctag

SEQ ID NO: 80 - DNA, 570 bp, Acremonium cellulolyticus
tcaatcacgacgagccagactgggacgaacaacggctactactactcgttctggaccaacggcggaggagaagtcacctacacaaatggtgacaatggcgaatacagcgtgacctgggtcaattgcggtgactttacatctggcaagggctggaatccagctaatgcacagactgtcacctactctggagaatttaatacctctggaaacgcttatctcgccgtttacggttggacaactgatcctcttgtcgaatactacatcctggagtcctacggttactataacccatcatctggccttacattacttggccaggttactagcgatggtggtacgtacgatatctactcaacacagcgtgtcgaccaaccctccatcgagggaacttccaccttcaatcagtactggtcggttcgcacagagaagcgagtcggcggaactgtcaccacggccaaccactttgcagcatggaaggcacttggacttgaaatgggtacttataactatatgattgtgtctacagaaggctacgagagcagtggttctagtaccatcaccgtgtcctag

SEQ ID NO: 81 - DNA, 570 bp, Acremonium cellulolyticus; identical to SEQ ID NO: 68

SEQ ID NO: 82 - DNA, 570 bp, Acremonium cellulolyticus
tcaatcacgacgagccagactgggacgaacaacggctactactactcgttctggaccaacggcggaggagaagtcacctacacaaatggtgacaatggcgaatacagcgtgacctgggtcaattgcggtgactttacatctggcaagggctggaatccagctaatgcacagactgtcacctactctggagaatttaatacctctggaaacgcttatctcgccgtttacggttggacaactgatcctcttgtcgaatactacatcctggagtcctacggtacatataacccatcatctggccttacattacttggccaggttactagcgatggtggtacgtacgatatctactcaacacagcgtgtcgaccaaccctccatcgagggaacttccaccttcaatcagtacgcgtcggttcgcacagagaagcgagtcggcggaactgtcaccacggccaaccactttgcagcatggaaggcacttggacttgaaatgggtacttataactatatgattgtgtctacagaaggctacgagagcagtggttctagtaccatcaccgtgtcctag

SEQ ID NO: 83 - DNA, 570 bp, Acremonium cellulolyticus
tcaatcacgacgagccagactgggacgaacaacggctactactactcgttctggaccaacggcggaggagaagtcacctacacaaatggtgacaatggcgaatacagcgtgacctgggtcaattgcggtgactttacatctggcaagggctggaatccagctaatgcacagactgtcacctactctggagaatttaatacctctggaaacgcttatctcgccgtttacggttggacaactgatcctcttgtcgaatactacatcctggagtcctacggtacatataacccatcatctggccttacattacttggccaggttactagcgatggtggtacgtacgatatctactcaacacagcgtgtcgaccaaccctccatcgagggaacttccaccttcaatcagtactggtcggttcgcacagagaagcgagtcggcggaactgtcaccacggccaaccactttgcagcatggaaggcacttggacttgaaatgggtacttataactatatgattgtgtctacagaaggcgcggagagcagtggttctagtaccatcaccgtgtcctag

SEQ ID NO: 84 - DNA, 570 bp, Acremonium cellulolyticus
tgcatcacgacgagccagactgggacgcacaacggctactactactcgttctggaccaacggcggaggagaagtcaccatgtgtttaggtcctggtggcgaatacagcgtgacctgggtcaattgcggtgcgtttacatctggcaagggctggaatccagctaatgcccagactgtcacctactctggagaatttaatcccaatggaaacgcttatctcgccgtttacggttggacaactgatcctcttgtcgaatactacatcctggagtcctacggtacatataacccatcatctggccttacattacttggccaggttactagcgatggtggtacgtacgatatctactcaacacagcgtgtcgaccaaccctccatcgagggaacttccaccttcaatcagtactggtcggttcgcacagagaagcgagtcggcggaactgtcaccacggccaaccactttgcagcatggaaggcacttggacttgaaatgggtacttataactatatgattgtgtctacagaaggctacgagagcagtggttctagtaccatcaccgtgtcctag

SEQ ID NO: 85 - DNA, 570 bp, Acremonium cellulolyticus
tgcatcacgacgagccagactgggacgcacaacggctactactactcgttctggaccaacggcggaggagaagtcaccatgtgtttaggtcctggtggcgaatacagcgtgacctgggtcaattgcggtgactttgcgtctggcaagggctggaatccagctaatgcccagactgtcacctactctggagaatttaatcccaatggaaacgcttatctcgccgtttacggttggacaactgatcctcttgtcgaatactacatcctggagtcctacggtacatataacccatcatctggccttacattacttggccaggttactagcgatggtggtacgtacgatatctactcaacacagcgtgtcgaccaaccctccatcgagggaacttccaccttcaatcagtactggtcggttcgcacagagaagcgagtcggcggaactgtcaccacggccaaccactttgcagcatggaaggcacttggacttgaaatgggtacttataactatatgattgtgtctacagaaggctacgagagcagtggttctagtaccatcaccgtgtcctag

SEQ ID NO: 86 - DNA, 570 bp, Acremonium cellulolyticus
tgcatcacgacgagccagactgggacgcacaacggctactactactcgttctggaccaacggcggaggagaagtcaccatgtgtttaggtcctggtggcgaatacagcgtgacctgggtcaattgcggtgcgtttacatctggcaagggctggaatccagctaatgcccagactgtcacctactctggagaatttaatcccaatggaaacgcttatctcgccgtttacggttggacaactgatcctcttgtcgaatactacatcctggagtcctacggtacatataacccatcatctggccttacattacttggccaggttactagcgatggtggtacgtacgatatctactcaacacaggcggtcgaccaaccctccatcgagggaacttccaccttcaatcagtactggtcggttcgcacagagaagcgagtcggcggaactgtcaccacggccaaccactttgcagcatggaaggcacttggacttgaaatgggtacttataactatatgattgtgtctacagaaggctacgagagcagtggttctagtaccatcaccgtgtcctag

SEQ ID NO: 87 - protein, 229 aa, Trichoderma reesei
MVAFSSLICALTSIASTLAMPTGLEPESSVNVTERGMYDFVLGAHNDHRRRASINYDQNYQTGGQVSYSPSNTGFSVNWNTQDDFVVGVGWTTGSSAPINFGGSFSVNSGTGLLSVYGWSTNPLVEYYIMEDNHNYPAQGTVKGTVTSDGATYTIWENTRVNEPSIQGTATFNQYISVRNSPRTSGTVTVQNHFNAWASLGLHLGQMNYQVVAVEGWGGSGSASQSVSN

SEQ ID NO: 88 - protein, 178 aa, Trichoderma reesei
ASINYDQNYQTGGQVSYSPSNTGFSVNWNTQDDFVVGVGWTTGSSAPINFGGSFSVNSGTGLLSVYGWSTNPLVEYYIMEDNHNYPAQGTVKGTVTSDGATYTIWENTRVNEPSIQGTATFNQYISVRNSPRTSGTVTVQNHFNAWASLGLHLGQMNYQVVAVEGWGGSGSASQSVSN

SEQ ID NO: 89 - protein, 178 aa, Trichoderma reesei
ASINYDQNYQTGGQVSYSPSNTGFSVNWNTQDAFVVGVGWTTGSSAPINFGGSFSVNSGTGLLSVYGWSTNPLVEYYIMEDNHNYPAQGTVKGTVTSDGATYTIWENTRVNEPSIQGTATFNQYISVRNSPRTSGTVTVQNHFNAWASLGLHLGQMNYQVVAVEGWGGSGSASQSVSN

SEQ ID NO: 90 - protein, 178 aa, Trichoderma reesei
ASINYDQNYQTGGQVSYSPSNTGFSVNWNTQDDFAVGVGWTTGSSAPINFGGSFSVNSGTGLLSVYGWSTNPLVEYYIMEDNHNYPAQGTVKGTVTSDGATYTIWENTRVNEPSIQGTATFNQYISVRNSPRTSGTVTVQNHFNAWASLGLHLGQMNYQVVAVEGWGGSGSASQSVSN

SEQ ID NO: 91 - protein, 690 aa, Trichoderma reesei
ATGGTTGCCTTTTCCAGCCTCATCTGCGCTCTCACCAGCATCGCCAGTACTCTGGCGATGCCCACAGGCCTCGAGCCTGAGAGCAGTGTCAACGTCACAGAGCGTGGCATGTACGACTTTGTTCTTGGAGCTCACAATGATCATCGCCGTCGTGCTAGCATCAACTACGACCAAAACTACCAAACTGGCGGACAAGTCAGCTATTCGCCTTCCAACACTGGCTTCTCAGTGAACTGGAACACTCAAGATGACTTTGTTGTGGGCGTTGGTTGGACGACTGGATCTTCTGCTCCCATCAACTTTGGCGGCTCTTTTAGTGTCAACAGCGGAACTGGCCTGCTTTCCGTCTATGGCTGGAGCACCAACCCACTGGTTGAGTACTACATCATGGAGGACAACCACAACTACCCAGCACAGGGTACCGTCAAGGGAACCGTCACCAGCGACGGAGCCACTTACACCATCTGGGAGAATACCCGTGTCAACGAGCCTTCCATCCAGGGCACAGCGACCTTCAACCAGTACATTTCCGTGCGGAACTCGCCCAGGACCAGCGGAACTGTTACTGTGCAGAACCACTTCAATGCTTGGGCCTCGCTTGGCCTGCACCTTGGGCAGATGAACTACCAGGTTGTCGCTGTCGAAGGCTGGGGTGGTAGTGGTTCTGCCTCACAGAGTGTCAGCAACTAG

SEQ ID NO: 92 - protein, 537 aa, Trichoderma reesei
GCTAGCATCAACTACGACCAAAACTACCAAACTGGCGGACAAGTCAGCTATTCGCCTTCCAACACTGGCTTCTCAGTGAACTGGAACACTCAAGATGACTTTGTTGTGGGCGTTGGTTGGACGACTGGATCTTCTGCTCCCATCAACTTTGGCGGCTCTTTTAGTGTCAACAGCGGAACTGGCCTGCTTTCCGTCTATGGCTGGAGCACCAACCCACTGGTTGAGTACTACATCATGGAGGACAACCACAACTACCCAGCACAGGGTACCGTCAAGGGAACCGTCACCAGCGACGGAGCCACTTACACCATCTGGGAGAATACCCGTGTCAACGAGCCTTCCATCCAGGGCACAGCGACCTTCAACCAGTACATTTCCGTGCGGAACTCGCCCAGGACCAGCGGAACTGTTACTGTGCAGAACCACTTCAATGCTTGGGCCTCGCTTGGCCTGCACCTTGGGCAGATGAACTACCAGGTTGTCGCTGTCGAAGGCTGGGGTGGTAGTGGTTCTGCCTCACAGAGTGTCAGCAACTAG

SEQ ID NO: 93 - protein, 537 aa, Trichoderma reesei
GCTAGCATCAACTACGACCAAAACTACCAAACTGGCGGACAAGTCAGCTATTCGCCTTCCAACACTGGCTTCTCAGTGAACTGGAACACTCAAGATGCGTTTGTTGTGGGCGTTGGTTGGACGACTGGATCTTCTGCTCCCATCAACTTTGGCGGCTCTTTTAGTGTCAACAGCGGAACTGGCCTGCTTTCCGTCTATGGCTGGAGCACCAACCCACTGGTTGAGTACTACATCATGGAGGACAACCACAACTACCCAGCACAGGGTACCGTCAAGGGAACCGTCACCAGCGACGGAGCCACTTACACCATCTGGGAGAATACCCGTGTCAACGAGCCTTCCATCCAGGGCACAGCGACCTTCAACCAGTACATTTCCGTGCGGAACTCGCCCAGGACCAGCGGAACTGTTACTGTGCAGAACCACTTCAATGCTTGGGCCTCGCTTGGCCTGCACCTTGGGCAGATGAACTACCAGGTTGTCGCTGTCGAAGGCTGGGGTGGTAGTGGTTCTGCCTCACAGAGTGTCAGCAACTAG

SEQ ID NO: 94 - protein, 537 aa, Trichoderma reesei
GCTAGCATCAACTACGACCAAAACTACCAAACTGGCGGACAAGTCAGCTATTCGCCTTCCAACACTGGCTTCTCAGTGAACTGGAACACTCAAGATGACTTTGCGGTGGGCGTTGGTTGGACGACTGGATCTTCTGCTCCCATCAACTTTGGCGGCTCTTTTAGTGTCAACAGCGGAACTGGCCTGCTTTCCGTCTATGGCTGGAGCACCAACCCACTGGTTGAGTACTACATCATGGAGGACAACCACAACTACCCAGCACAGGGTACCGTCAAGGGAACCGTCACCAGCGACGGAGCCACTTACACCATCTGGGAGAATACCCGTGTCAACGAGCCTTCCATCCAGGGCACAGCGACCTTCAACCAGTACATTTCCGTGCGGAACTCGCCCAGGACCAGCGGAACTGTTACTGTGCAGAACCACTTCAATGCTTGGGCCTCGCTTGGCCTGCACCTTGGGCAGATGAACTACCAGGTTGTCGCTGTCGAAGGCTGGGGTGGTAGTGGTTCTGCCTCACAGAGTGTCAGCAACTAG

(19) United States
(12) Patent Application Publication
(10) Pub. No.: US 2020/0002695 A1
(43) Pub. Date: Jan. 2, 2020

(54) XYLANASE VARIANT AND ENZYME COMPOSITION FOR DECOMPOSING BIOMASS

(71) Applicants: Toray Industries, Inc., Tokyo (JP); National Institute of Advanced Industrial Science and Technology, Tokyo (JP)

(72) Inventors: Natsuko Murakami, Kamakura-shi (JP); Koji Kobayashi, Kamakura-shi (JP); Hiroyuki Kurihara, Otsu-shi (JP); Katsushige Yamada, Kamakura-shi (JP); Masahiro Watanabe, Higashihiroshima-shi (JP); Keiko Uechi, Higashihiroshima-shi (JP); Tamotsu Hoshino, Higashihiroshima-shi (JP)

(21) Appl. No.: 16/485,205

(22) PCT Filed: Feb. 23, 2018
(86) PCT No.: PCT/JP2018/006793

§ 371 Date: Aug. 12, 2019

(30) Foreign Application Priority Data
Feb. 23, 2017 (JP) 2017-032346

(51) Int. Cl. C12N 9/24

(52) U.S. Cl. CPC C12N 9/2482; C12Y 302/01008

(57) ABSTRACT

A xylanase variant includes an amino acid sequence derived from an amino acid sequence of a xylanase from a filamentous fungus wherein one or more amino acid residues at the positions selected from the positions corresponding to position 78, position 80, position 117, position 155, position 169, and position 203 in the amino acid sequence of SEQ ID NO: 1 are substituted, the xylanase variant having a xylanase activity.

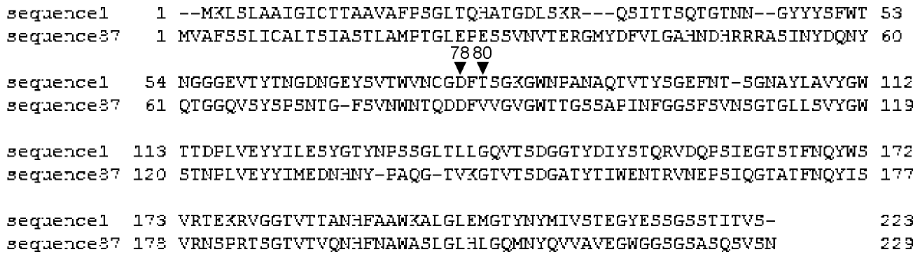

### TECHNICAL FIELD

This disclosure relates to novel xylanase variants and enzyme compositions for decomposing biomass comprising the same.

### BACKGROUND

There are various techniques for saccharification of cellulose, and the main stream of their development is an enzymic saccharification method that requires less energy consumption and achieves a high sugar yield. Cellulose is much contained in herbaceous plants and arboreous plants, and these plants are collectively referred to as cellulose-containing biomass. Cellulose-containing biomass contains not only cellulose, but also hemicelluloses such as xylan and arabinan, and lignin. Xylan has β-1,4-bonded D-xylose as the main chain, and this main chain may be partially modified with O-acetyl, β-arabinofuranosyl, glucuronic acid, or phenolic acid (M. P. Coughlan et al., Biotechnol. Appl. Biochem. 17, 259-289 (1993)). Xylanase is one of the important enzymes which decompose cellulose-containing biomass through acting on the β-1,4-bonded xylose main chain.

Xylanase is classified into Glycoside Hydrolase Family 10 (GH10) and Glycoside Hydrolase Family 11 (GH11) by the homology of the amino acid sequence (P. M. Coutinho et al., Recent Advances in Carbohydrate Bioengineering, 246, 3-12 (1999)). GH10 xylanase generally has a molecular weight of 30 kDa or more, whereas it is said that generally the molecular weight of GH11 xylanase is approximately 20 kDa and is relatively small (Beaugrand et al., Carbohydr. Res. 339, 2529-2540 (2004)). Structural analysis has been performed on some of GH11 xylanases, and it is reported that glutamic acid, aromatic amino acid, and charged amino acid located at specified positions on the surface of the active site of the enzyme are important for the enzyme activity (Tariq A. Tahir et al., The Journal of Biological Chemistry, 277, 46, 44035-44043 (2002)).

Filamentous fungi are known as microorganisms that decompose a wide range of cellulose-based biomasses. It is known that cellulase produced in a culture medium by Acremonium cellulolyticus (presently also referred to as Talaromyces cellulolyticus) can provide a higher glucose yield in decomposition of cellulose-containing biomass than cellulase produced by Trichoderma reesei (Fujii et al., Biotechnol. Biofuels. 2, 24 (2009)). In recent years, seven types of xylanases have been cloned from Acremonium cellulolyticus, and the wild-type enzymes thereof have been functionally analyzed (Watanabe et al., AMB Express. 4, 27). Watanabe et al. have reported that, among the seven xylanases, XylC is expressed at the least level in Acremonium cellulolyticus, but has the highest xylanolytic activity.

Xylanase is commercially utilized in the fields of the paper-making industry, the food industry, the pharmaceutical industry and the like, and also used in the production of oligosaccharide. However, there is a problem that, when oligosaccharide is produced by hydrolysis by xylanase, xylotriose is generally hydrolyzed to generate xylobiose and xylose, which is a monosaccharide, consequently reducing the oligosaccharide yield.

### SUMMARY

We found that hydrolysis of xylotriose can be suppressed and the oligosaccharide yield can be improved in hydrolysis by a xylanase having an amino acid sequence derived from a xylanase from filamentous fungus wherein one or more amino acid residues at the positions selected from the positions corresponding to position 78, position 80, position 117, position 155, position 169, and position 203 in the amino acid sequence of SEQ ID NO: 1 are each substituted with a different amino acid residue. We thus provide:

[1] A xylanase variant derived from an amino acid sequence of a xylanase from a filamentous fungus, comprising an amino acid sequence wherein one or more amino acid residues at the positions selected from the positions corresponding to position 78, position 80, position 117, position 155, position 169, and position 203 in the amino acid sequence of SEQ ID NO: 1 are each substituted with a different amino acid residue, the xylanase variant having a xylanase activity whereby the productivity of xylotriose from xylan is improved compared with the original xylanase wherein one or more amino acid residues are not substituted.

[2] The xylanase variant according to [1], comprising an amino acid sequence wherein the amino acid residue at the position corresponding to position 78 in the amino acid sequence of SEQ ID NO: 1 is substituted with alanine, glycine, valine, leucine, or isoleucine.

[3] The xylanase variant according to [1], comprising an amino acid sequence wherein the amino acid residue at the position corresponding to position 80 in the amino acid sequence of SEQ ID NO: 1 is substituted with alanine, glycine, leucine, or isoleucine.

[4] The xylanase variant according to [1], comprising an amino acid sequence wherein the amino acid residue at the position corresponding to position 117 in the amino acid sequence of SEQ ID NO: 1 is substituted with serine, threonine, or asparagine.

[5] The xylanase variant according to [1], comprising an amino acid sequence wherein the amino acid residue at the position corresponding to position 155 in the amino acid sequence of SEQ ID NO: 1 is substituted with alanine, glycine, valine, leucine, or isoleucine.

[6] The xylanase variant according to [1], comprising an amino acid sequence wherein the amino acid residue at the position corresponding to position 169 in the amino acid sequence of SEQ ID NO: 1 is substituted with alanine, glycine, valine, leucine, or isoleucine.

[7] The xylanase variant according to [1], comprising an amino acid sequence wherein the amino acid residue at the position corresponding to position 203 in the amino acid sequence of SEQ ID NO: 1 is substituted with tryptophan, phenylalanine, or tyrosine.

[8] The xylanase variant according to [1], [2], or [5], comprising an amino acid sequence wherein the amino acid residue at the position corresponding to position 78 in the amino acid sequence of SEQ ID NO: 1 is substituted with alanine and wherein the amino acid residue at the position corresponding to position 155 in the amino acid sequence of SEQ ID NO: 1 is substituted with alanine.

[9] The xylanase variant according to any one of [1] to [8], wherein the original xylanase is a filamentous fungus-derived xylanase belonging to Glycoside Hydrolase Family 11.

[10] The xylanase variant according to [1], comprising any one of the amino acid sequences of following (a) to (c), and having a xylanase activity:

(a) any one of the amino acid sequences shown in SEQ ID NOs: 3 to 9, 89, and 90;

(b) an amino acid sequence derived from any one of the amino acid sequences shown in SEQ ID NOs: 3 to 9, 89, and 90, wherein the amino acids substituted at the positions corresponding to position 78, position 80, position 117, position 155, position 169, and position 203 in the amino acid sequence of SEQ ID NO: 1 are not mutated, and wherein one to several amino acids are deleted, substituted, inserted, or added at positions of amino acids other than the substituted amino acids; or

(c) an amino acid sequence derived from any one of the amino acid sequences shown in SEQ ID NOs: 3 to 9, 89, and 90, wherein the amino acids substituted at the positions corresponding to position 78, position 80, position 117, position 155, position 169, and position 203 in the amino acid sequence of SEQ ID NO: 1 are not mutated, and wherein the amino acid sequence except the substituted amino acids has an amino acid identity of 90% or more to any one of the amino acid sequences.

[11] The xylanase variant according to any one of [1] to [10], wherein the amino acid residues at the positions corresponding to position 35, position 44, position 61, position 62, position 63, position 65, position 66, position 101, and position 102 in the amino acid sequence of SEQ ID NO: 1 are further each substituted with a different amino acid residue.

[12] The xylanase variant according to [11], wherein:

the amino acid residue at the position corresponding to position 35 in the amino acid sequence of SEQ ID NO: 1 is substituted with cysteine,

the amino acid residue at the position corresponding to position 44 in the amino acid sequence of SEQ ID NO: 1 is substituted with histidine,

the amino acid residue at the position corresponding to position 61 in the amino acid sequence of SEQ ID NO: 1 is substituted with methionine,

the amino acid residue at the position corresponding to position 62 in the amino acid sequence of SEQ ID NO: 1 is substituted with cysteine,

the amino acid residue at the position corresponding to position 63 in the amino acid sequence of SEQ ID NO: 1 is substituted with leucine,

the amino acid residue at the position corresponding to position 65 in the amino acid sequence of SEQ ID NO: 1 is substituted with proline,

the amino acid residue at the position corresponding to position 66 in the amino acid sequence of SEQ ID NO: 1 is substituted with glycine,

the amino acid residue at the position corresponding to position 101 in the amino acid sequence of SEQ ID NO: 1 is substituted with proline, and

the amino acid residue at the position corresponding to position 102 in the amino acid sequence of SEQ ID NO: 1 is substituted with asparagine.

[13] The xylanase variant according to [12], comprising any one of the amino acid sequences of following (d) to (f), and having a xylanase activity:

(d) any one of the amino acid sequences shown in SEQ ID NOs: 18 to 20;

(e) an amino acid sequence derived from any one of the amino acid sequences shown in SEQ ID NOs: 18 to 20, wherein the amino acids substituted at the positions corresponding to position 35, position 44, position 61, position 62, position 63, position 65, position 66, position 78, position 80, position 101, position 102, and position 155 in the amino acid sequence of SEQ ID NO: 1 are not mutated, and wherein one to several amino acids are deleted, substituted, inserted, or added at positions of amino acids other than the substituted amino acids; or

(f) an amino acid sequence derived from any one of the amino acid sequences shown in SEQ ID NOs: 18 to 20, wherein the amino acids substituted at the positions corresponding to position 35, position 44, position 61, position 62, position 63, position 65, position 66, position 78, position 80, position 101, position 102, and position 155 in the amino acid sequence of SEQ ID NO: 1 are not mutated, and wherein the amino acid sequence except the substituted amino acids has an amino acid identity of 90% or more to any one of the amino acid sequences.

[14] An enzyme composition comprising the xylanase variant according to any one of [1] to [13].

This disclosure incorporates by reference the contents disclosed in Japanese Patent Application No. 2017-32346, to which the present application claims priority.

We provide a xylanase variant wherein hydrolysis of xylotriose by the variant is suppressed. The xylanase variant has an effect of improving the oligosaccharide yield since the amount of production of xylotriose is improved and the amount of production of xylose is decreased in the hydrolysis of xylan by the xylanase variant. Accordingly, the xylanase variant and an enzyme composition comprising the xylanase variant can be suitably used to produce oligosaccharide from cellulose-containing biomass.

### BRIEF DESCRIPTION OF THE DRAWING

The Drawing shows the result of alignment of the amino acid sequences shown in SEQ ID NO: 1 and SEQ ID NO: 87. In the Drawing, 78 and 80 correspond to position 78 and position 80 in SEQ ID NO: 1, respectively. In SEQ ID NO: 87, the positions corresponding to these are positions 84 and 86 respectively. In the xylanase variant, the amino acids at these positions can be substituted.

### DETAILED DESCRIPTION

Our variants and compositions will be described in detail, but this disclosure is not limited to them.

### (1) Xylanase Variant

A “xylanase” refers to an enzyme having an activity of hydrolyzing hemicellulose by acting on the β-1,4-bonded main chain of xylose (xylanase activity), and the enzyme is classified into EC 3.2.1.8. Xylanases are classified into two types: Glycoside Hydrolase Family 10 (GH10) and Glycoside Hydrolase Family 11 (GH11), and the enzymes classified into GH11 have a β jelly roll structure. The “xylanase activity” can be measured using β-1,4-bonded D-xylose as a substrate, preferably using, as a substrate, Birchwood xylan sold as a reagent. Whether xylan as a substrate is or is not decomposed can be verified by measuring the amount of the reducing sugar comprised in a reaction solution after the reaction. The amount of reducing sugar can be measured using the dinitrosalicylic acid method (DNS method), and the method described in Bailey et al., “Interlaboratory testing of methods for assay of xylanase activity,” J. Biotechnol. 23, 257-270 can preferably be used. The conditions under which the activity is measured are not particularly limited as long as the activity of xylanase can be measured by the method described above. Preferable temperature conditions for measurement of the activity are 20° C. to 90° C., preferably 40° C. to 75° C., the pH is preferably 4 to 9, more preferably pH 5 to 7, and the reaction time is preferably one second to 600 minutes, most preferably one minute to 60 minutes. In addition, the xylan used as a substrate for measurement of the activity is preferably 0.1% by weight to 10% by weight, most preferably 0.5% by weight to 2% by weight.

The “original xylanase” refers to a xylanase consisting of an amino acid sequence before introduction of substitution mutations at specified amino acid positions in the xylanase variant. Specifically, it refers to a xylanase derived from a filamentous fungus-derived xylanase wherein, assuming the amino acid sequence of SEQ ID NO: 1 as a reference sequence, amino acid residues at any of the positions corresponding to position 78, position 80, position 117, position 155, position 169, and position 203 in the reference sequence are not substituted with a different amino acid residue. Examples include, but are not limited to, a filamentous fungus-derived wild-type xylanase. A “wild-type xylanase” refers to an enzyme having the original activity as a xylanase. Generally, wild-type xylanases are proteins encoded by wild-type xylanase genes existing in the genomes of various biological species. The biological species from which wild-type xylanases are derived are not limited, but filamentous fungus-derived xylanases belonging to Glycoside Hydrolase Family 11 are preferred. In addition, the original xylanase may be, for example, a filamentous fungus-derived variant xylanase. In this example, the xylanase variant is a xylanase derived from the variant xylanase further comprising mutations in which the amino acid residues at any of the positions corresponding to position 78, position 80, position 117, position 155, position 169, and position 203 in the reference sequence described above are each substituted with a different amino acid residue.

A filamentous fungus refers to a fungus that forms fungal filaments, and examples of filamentous fungi that can be utilized include, but are not limited to, Trichoderma, Aspergillus, Cellulomonas, Clostridium, Streptomyces, Humicola, Acremonium, Irpex, Mucor, Talaromyces, Thermomyces, Paecilomyces and the like. For example, a xylanase derived from Acremonium cellulolyticus, Trichoderma reesei, Trichoderma harzianum, Aspergillus niger, Aspergillus kawachii, Thermomyces lanuginosus, Paecilomyces variotii, Paecilomyces sp. J18, Cellulomonas flavigena, Cellulomonas bogoriensis, Clostridium thermocellum, Streptomyces sp. S38, Humicola insolens, Talaromyces leycettanus or the like (without limitation thereto) can be utilized.

In addition, a xylanase derived from a variant strain obtained from the filamentous fungus described above by performing mutagenesis using a mutagenesis agent, ultraviolet irradiation or the like can be utilized. Various filamentous fungus-derived xylanases have been isolated and identified, and gene information and the like on the xylanases are disclosed in known databases such as GenBank. For example, xylanases from Acremonium cellulolyticus are registered as AB847990, AB847991, AB847992, AB847993, AB847994, AB847995, and AB847996 in GenBank, a xylanase from Trichoderma reesei is registered as AAB29346 in GenBank, xylanases from Trichoderma harzianum are registered as ACF40831 and KM001857 in GenBank, xylanases from Aspergillus niger are registered as AM270980, AFK10490, and AAA99065 in GenBank, xylanases from Aspergillus kawachii are registered as D38070, AAC60542, and BAA07264 in GenBank, xylanases from Thermomyces lanuginosus are registered as HM123759, AEH57194, and AAB94633 in GenBank, a xylanase from Paecilomyces variotii is registered as AAS31744 in GenBank, xylanases from Paecilomyces sp. J18 are registered as FJ593504 and ACS26244 in GenBank, xylanases from Cellulomonas flavigena are registered as CAJ57849, ADG73165, AAK15536, and AF338352 in GenBank, a xylanase from Streptomyces sp. S38 is registered as CAA67143 in GenBank, and xylanases from Humicola insolens are registered as CAA53632, AHC72381, and AJF98581 in GenBank. This gene information and the like can be utilized.

The “original xylanase” belonging to Glycoside Hydrolase Family 11 is preferably derived from Acremonium or Trichoderma, more preferably derived from Acremonium cellulolyticus or Trichoderma reesei. Specific examples include xylanases comprising or consisting of the amino acid sequence shown in SEQ ID NO: 1 or SEQ ID NO: 87.

In addition, the “original xylanase” comprises a portion of the “original xylanase” described above as long as it retains a xylanase activity. “A portion of the original xylanase” may consist of a fragment of a xylanase formed by removing any partial region from the xylanase as long as the fragment retains at least 40% or more, 50% or more, 60% or more, 70% or more, 80% or more, 90% or more, 95% or more, or 99% or more of the activity of the original xylanase. Examples of such fragments include those xylanases from which the signal peptide region has been removed. Examples of signal peptides include the region represented by the amino acid sequence from position 1 to position 34 in the amino acid sequence shown in SEQ ID NO: 1 and the region represented by the amino acid sequence from position 1 to position 51 in the amino acid sequence shown in SEQ ID NO: 87. The amino acid sequence derived from the amino acid sequence shown in SEQ ID NO: 1 wherein the sequence of the signal peptide has been removed is shown as SEQ ID NO: 2. Furthermore, the amino acid sequence derived from the amino acid sequence shown in SEQ ID NO: 87 wherein the sequence of the signal peptide has been removed is shown as SEQ ID NO: 88. “A portion of the original xylanase” is preferably a polypeptide fragment comprising or consisting of the amino acid sequence shown in SEQ ID NO: 2 or SEQ ID NO: 88.

The “xylanase variant” means a protein having an amino acid sequence of the “original xylanase” derived from any filamentous fungus wherein amino acid residues at one or more positions selected from the positions corresponding to position 78, position 80, position 117, position 155, position 169, and position 203 in the amino acid sequence of SEQ ID NO: 1 are each substituted with a different amino acid residue, the protein having a xylanase activity. The xylanase variant preferably comprises a substitution of the amino acid residue at the positions corresponding to position 78, position 80, position 117, position 155, position 169, or position 203 in the amino acid sequence of SEQ ID NO: 1. The xylanase variant more preferably comprises a substitution of the amino acid residue at the position corresponding to position 78 or position 80 in the amino acid sequence of SEQ ID NO: 1, more preferably comprises substitutions of the amino acid residues at the two positions corresponding to position 78 and position 155 in the amino acid sequence of SEQ ID NO: 1.

The “xylanase variant” comprises amino acid substitutions at the positions described above, and accordingly has a xylanase activity whereby the amount of production of xylotriose from xylan is improved compared to the original xylanase in which the amino acid residues are not substituted.

The amino acid positions identified by “the positions corresponding to position 78, position 80, position 117, position 155, position 169, and position 203 in the amino acid sequence of SEQ ID NO: 1” in the amino acid sequence of the xylanase described above can be determined by the technique including Procedure 1 to Procedure 3 described below.

Procedure 1: In the amino acid sequence of SEQ ID NO: 1, the initiator methionine is defined as position 1. The positions that follow in the amino acid sequence are numbered as position 2, 3, 4, . . . , and thereby each position is defined.

Procedure 2: Next, the amino acid positions in the amino acid sequence of the original xylanase which is the subject to be mutated corresponding to position 78, position 80, position 117, position 155, position 169, and position 203 in the amino acid sequence shown in SEQ ID NO: 1 are determined. The corresponding amino acid positions can be clarified by aligning the amino acid sequence of the original xylanase to be mutated to the amino acid sequence of SEQ ID NO: 1 such that the identity and similarity between the two sequences become maximum. Such an operation is called alignment of amino acid sequence. As an alignment tool, the Parallel Editor (Multiple Allignment) in Genetyx, a program included in ClustalW, is used with default parameters. By alignment between amino acid sequences having different lengths, those skilled in the art can clarify the amino acid positions in the amino acid sequence of the original xylanase which is the subject to be mutated corresponding to position 78, position 80, position 117, position 155, position 169, and position 203 in the amino acid sequence represented by SEQ ID NO: 1.

Procedure 3: The positions corresponding to position 78, position 80, position 117, position 155, position 169, and position 203 in the amino acid sequence of SEQ ID NO: 1 in the alignment analysis described above are determined as the “positions corresponding to position 78, position 80, position 117, position 155, position 169, and position 203 in the amino acid sequence of SEQ ID NO: 1” in the amino acid sequence of the “xylanase,” which is the subject to be mutated.

When the original xylanase which is the subject to be mutated comprises mutations such as deletion, substitution, addition, or insertion of an amino acid at positions other than the “positions corresponding to position 78, position 80, position 117, position 155, position 169, and position 203 in the amino acid sequence of SEQ ID NO: 1” described above, or in a portion of the original xylanase, the “positions corresponding to position 78, position 80, position 117, position 155, position 169, and position 203 in the amino acid sequence of SEQ ID NO: 1” may not be the 78th, 80th, 117th, 155th, 169th, and 203rd, respectively, as counted from the N-terminus of the original xylanase which is the subject to be mutated or a portion thereof. Also in these examples, the positions determined by the technique described above are the “positions corresponding to position 78, position 80, position 117, position 155, position 169, and position 203 in the amino acid sequence of SEQ ID NO: 1.”

A substitution of the amino acids at the corresponding positions in the original xylanase which is the subject to be mutated may be a substitution with a different amino acid, and the substitution preferably comprises substitution with the following amino acids, without particular limitation, at the respective positions:

the position corresponding to position 78 in the amino acid sequence of SEQ ID NO: 1: alanine, glycine, valine, leucine, or isoleucine, preferably alanine;

the position corresponding to position 80 in the amino acid sequence of SEQ ID NO: 1: alanine, glycine, leucine, or isoleucine, preferably alanine;

the position corresponding to position 117 in the amino acid sequence of SEQ ID NO: 1: serine, threonine, or asparagine, preferably asparagine;

the position corresponding to position 155 in the amino acid sequence of SEQ ID NO: 1: alanine, glycine, valine, leucine, or isoleucine, preferably alanine;

the position corresponding to position 169 in the amino acid sequence of SEQ ID NO: 1: alanine, glycine, valine, leucine, or isoleucine, preferably alanine;

the position corresponding to position 203 in the amino acid sequence of SEQ ID NO: 1: tryptophan, phenylalanine, or tyrosine, preferably tryptophan.

In one aspect, the xylanase variant comprises or consists of a polypeptide having an amino acid sequence or a portion thereof derived from the amino acid sequence of SEQ ID NO: 1 wherein the amino acids at one or more positions selected from position 78, position 80, position 117, position 155, position 169, and position 203 are substituted, and having a xylanase activity.

In one aspect other than those described above, the xylanase variant comprises or consists of a polypeptide having an amino acid sequence or a portion thereof derived from the amino acid sequence of SEQ ID NO: 87 wherein the amino acids at one or two positions selected from the positions corresponding to position 78 and/or position 80 in SEQ ID NO: 1 are substituted, and having a xylanase activity.

Examples of the “a portion thereof” include a polypeptide obtained by removing the signal peptide region described above from the polypeptide of the xylanase variant. More specific examples of such xylanase variants include the following:

a xylanase variant derived from the amino acid sequence of SEQ ID NO: 1 comprising a mutation in which the aspartic acid at position 78 is substituted with alanine, the xylanase variant comprising or consisting of the amino acid sequence of SEQ ID NO: 3 (the amino acid sequence of SEQ ID NO: 3 does not comprise the signal peptide region described above);

a xylanase variant derived from the amino acid sequence of SEQ ID NO: 1 comprising a mutation in which the threonine at position 80 is substituted with alanine, the xylanase variant comprising or consisting of the amino acid sequence of SEQ ID NO: 4 (the amino acid sequence of SEQ ID NO: 4 does not comprise the signal peptide region described above);

a xylanase variant derived from the amino acid sequence of SEQ ID NO: 1 comprising a mutation in which the leucine at position 117 is substituted with asparagine, the xylanase variant comprising or consisting of the amino acid sequence of SEQ ID NO: 5 (the amino acid sequence of SEQ ID NO: 5 does not comprise the signal peptide region described above);

a xylanase variant derived from the amino acid sequence of SEQ ID NO: 1 comprising a mutation in which the arginine at position 155 is substituted with alanine, the xylanase variant comprising or consisting of the amino acid sequence of SEQ ID NO: 6 (the amino acid sequence of SEQ ID NO: 6 does not comprise the signal peptide region described above);

a xylanase variant derived from the amino acid sequence of SEQ ID NO: 1 comprising a mutation in which the glutamine at position 169 is substituted with alanine, the xylanase variant comprising or consisting of the amino acid sequence of SEQ ID NO: 7 (the amino acid sequence of SEQ ID NO: 7 does not comprise the signal peptide region described above);

a xylanase variant derived from the amino acid sequence of SEQ ID NO: 1 comprising a mutation in which the asparagine at position 203 is substituted with tryptophan, the xylanase variant comprising or consisting of the amino acid sequence of SEQ ID NO: 8 (the amino acid sequence of SEQ ID NO: 8 does not comprise the signal peptide region described above);

a xylanase variant derived from the amino acid sequence of SEQ ID NO: 1 comprising mutations in which the amino acids at both the positions of position 78 and position 155 are each substituted with alanine, the xylanase variant comprising or consisting of the amino acid sequence of SEQ ID NO: 9 (the amino acid sequence of SEQ ID NO: 9 does not comprise the signal peptide region described above).

A xylanase variant derived from the amino acid sequence of SEQ ID NO: 87 comprising a mutation in which the aspartic acid at the position corresponding to position 78 in the amino acid sequence of SEQ ID NO: 1 is substituted with alanine, the xylanase variant comprising or consisting of the amino acid sequence of SEQ ID NO: 89 (the amino acid sequence of SEQ ID NO: 89 does not comprise the signal peptide region described above);

a xylanase variant derived from the amino acid sequence of SEQ ID NO: 87 comprising a mutation in which the valine at the position corresponding to position 80 in the amino acid sequence of SEQ ID NO: 1 is substituted with alanine, the xylanase variant comprising or consisting of the amino acid sequence of SEQ ID NO: 90 (the amino acid sequence of SEQ ID NO: 90 does not comprise the signal peptide region described above).

The xylanase variant or a portion thereof also comprises a protein having an amino acid sequence wherein the amino acids substituted (if any) at the positions corresponding to position 78, position 80, position 117, position 155, position 169, and position 203 in the amino acid sequence of SEQ ID NO: 1 described above are not mutated, and wherein one or several amino acids are deleted, substituted, added, or inserted, the protein having a xylanase activity. The range of “one or several” is not particularly limited, and is, for example, within 10, more preferably within 5, particularly preferably within 4, or 1 or 2. In addition, the substitution of the amino acids other than the amino acids substituted (if any) at the positions corresponding to position 78, position 80, position 117, position 155, position 169, and position 203 in the amino acid sequence of SEQ ID NO: 1 described above may be, for example, a conservative amino acid substitution. A “conservative amino acid substitution” refers to a substitution between amino acids similar in nature such as electric charge, the side chain, polarity, or aromaticity. For example, a basic amino acid (arginine, lysine, histidine) can be substituted with a basic amino acid other than the original one, an acidic amino acid (aspartic acid, glutamic acid) can be substituted with an acidic amino acid other than the original one, an uncharged polar amino acid (glycine, asparagine, glutamine, serine, threonine, cysteine, tyrosine) can be substituted with an uncharged polar amino acid other than the original one, a non-polar amino acid (leucine, isoleucine, alanine, valine, proline, phenylalanine, tryptophan, methionine) can be substituted with a non-polar amino acid other than the original one, a branched chain amino acid (leucine, valine, isoleucine) can be substituted with a branched chain amino acid other than the original one, and an aromatic amino acid (phenylalanine, tyrosine, tryptophan, histidine) can be substituted with an aromatic amino acid other than the original one.

The xylanase variant or a portion thereof also comprises an amino acid sequence wherein the amino acids substituted (if any) at the positions corresponding to position 78, position 80, position 117, position 155, position 169, and position 203 in the amino acid sequence of SEQ ID NO: 1 described above are not mutated, and wherein the amino acid sequence has an amino acid identity of preferably 90% or more, more preferably 91%, 92%, 93%, 94%, 95%, 96%, 97%, 98%, 99%, or the same as or more than these, to the amino acid sequence of the xylanase variant or a portion thereof except the substituted amino acids, as calculated using BLAST (Basic Local Alignment Search Tool at the National Center for Biological Information in the U.S.A.) and the like (with, for example, default parameters, i.e., initial setting parameters). More preferably, the xylanase variant or a portion thereof comprises a protein consisting of an amino acid sequence having the preferable amino acid identity or the more preferable amino acid identity, the protein having a xylanase activity. Additionally, the xylanase variant or a portion thereof also comprises a protein comprising, more preferably consisting of, an amino acid sequence wherein the amino acids substituted (if any) at the positions in SEQ ID NO: 87 corresponding to position 78 and/or position 80 in the amino acid sequence of SEQ ID NO: 1 are not mutated, and wherein the amino acid sequence has an amino acid identity of preferably 90% or more, more preferably 91%, more preferably 92%, more preferably 93%, more preferably 94%, more preferably 95%, more preferably 96%, more preferably 97%, more preferably 98%, more preferably 99%, more preferably the same as or more than these, to the amino acid sequence of the xylanase variant or a portion thereof except the substituted amino acids, as calculated using BLAST and the like (with, for example, default parameters, i.e., initial setting parameters), the protein having a xylanase activity. An “amino acid identity” means a ratio (percentage) of the identical amino acids and similar amino acid residues to all overlapping amino acid residues in the optimal alignment in which two amino acid sequences are aligned with a gap introduced or not introduced. An amino acid identity can be determined using a method well-known to those skilled in the art, a sequence analysis software and the like (for example, a known algorithm such as BLAST or FASTA).

The xylanase variant may have an additional peptide or protein added to the N-terminus and/or C-terminus. Examples of such peptides or proteins include those comprising methionine as the translation initiation site, a secretion signal sequence, a transport protein, a binding protein, a tag peptide for purification, a heterologous hydrolase, a fluorescent protein and the like. As to such peptides or proteins, those skilled in the art can select, depending on the purpose, a peptide or protein having a function to be added, and add the peptide or protein to the xylanase variant.

The xylanase variant may comprise not only amino acid substitutions at one or more positions selected from the positions corresponding to position 78, position 80, position 117, position 155, position 169, and position 203 in the amino acid sequence of SEQ ID NO: 1, but also amino acid substitutions at one or more positions selected from the positions corresponding to position 35, position 44, position 61, position 62, position 63, position 65, position 66, position 101, and position 102 in the amino acid sequence of SEQ ID NO: 1. The amount of production of xylotriose can be further increased when xylanase variant comprises amino acid substitutions at these positions. The xylanase variant preferably comprises not only amino acid substitutions at one or more positions selected from the positions corresponding to position 78, position 80, position 117, position 155, position 169, and position 203 in the amino acid sequence of SEQ ID NO: 1, but also substitutions of the amino acid residues at the positions corresponding to position 35, position 44, position 61, position 62, position 63, position 65, position 66, position 101, and position 102.

The “positions corresponding to position 35, position 44, position 61, position 62, position 63, position 65, position 66, position 101, position 102 in the amino acid sequence of SEQ ID NO: 1” can be determined by a technique in accordance with Procedures 1 to 3 for determining the “positions corresponding to position 78, position 80, position 117, position 155, position 169, and position 203 in the amino acid sequence of SEQ ID NO: 1” described above, and the amino acid positions in the amino acid sequence of the xylanase described above corresponding to position 35, position 44, position 61, position 62, position 63, position 65, position 66, position 101, and position 102 in the amino acid sequence represented by SEQ ID NO: 1 can be determined by the alignment analysis described above.

A substitution of the amino acid at the corresponding positions may be a substitution with a different amino acid, and the substitution preferably comprises a substitution with the following amino acids, without particular limitation, at the respective positions:

the position corresponding to position 35 in the amino acid sequence of SEQ ID NO: 1: cysteine;

the position corresponding to position 44 in the amino acid sequence of SEQ ID NO: 1: histidine;

the position corresponding to position 61 in the amino acid sequence of SEQ ID NO: 1: methionine;

the position corresponding to position 62 in the amino acid sequence of SEQ ID NO: 1: cysteine;

the position corresponding to position 63 in the amino acid sequence of SEQ ID NO: 1: leucine;

the position corresponding to position 65 in the amino acid sequence of SEQ ID NO: 1: proline;

the position corresponding to position 66 in the amino acid sequence of SEQ ID NO: 1: glycine;

the position corresponding to position 101 in the amino acid sequence of SEQ ID NO: 1: proline;

the position corresponding to position 102 in the amino acid sequence of SEQ ID NO: 1: asparagine.

When a xylanase variant is expressed using a eukaryotic organism as a host, the amino acids at the positions corresponding to position 101 and position 102 may remain to be the original amino acids without being substituted. In some examples, the positions corresponding to position 101 and position 102 are comprised in an amino acid sequence to be glycosylated in a eukaryotic organism.

In one aspect, the xylanase variant comprises or consists of a polypeptide having an amino acid sequence or a portion thereof derived from the amino acid sequence of SEQ ID NO: 1 wherein the amino acids at one or more positions selected from position 78, position 80, position 117, position 155, position 169, and position 203 are substituted and the amino acids at position 35, position 44, position 61, position 62, position 63, position 65, position 66, position 101, and position 102 are substituted, the polypeptide having a xylanase activity. Examples of the “a portion thereof” include a polypeptide obtained by removing the signal peptide region described above from the polypeptide of the xylanase variant. More specific examples of such xylanase variants include the following:

a xylanase variant derived from the amino acid sequence of SEQ ID NO: 1 comprising a mutation in which the aspartic acid at position 78 is substituted with alanine, and comprising a mutation in which the serine at position 35 is substituted with cysteine, a mutation in which the asparagine at position 44 is substituted with histidine, a mutation in which the tyrosine at position 61 is substituted with methionine, a mutation in which the threonine at position 62 is substituted with cysteine, a mutation in which the asparagine at position 63 is substituted with leucine, a mutation in which the aspartic acid at position 65 is substituted with proline, a mutation in which the asparagine at position 66 is substituted with glycine, a mutation in which the threonine at position 101 is substituted with proline, and a mutation in which the serine at position 102 is substituted with asparagine, the xylanase variant comprising or consisting of the amino acid sequence of SEQ ID NO: 18 (the amino acid sequence of SEQ ID NO: 18 does not comprise the signal peptide region described above);

a xylanase variant derived from the amino acid sequence of SEQ ID NO: 1 comprising a mutation in which the threonine at position 80 is substituted with alanine, and comprising a mutation in which the serine at position 35 is substituted with cysteine, a mutation in which the asparagine at position 44 is substituted with histidine, a mutation in which the tyrosine at position 61 is substituted with methionine, a mutation in which the threonine at position 62 is substituted with cysteine, a mutation in which the asparagine at position 63 is substituted with leucine, a mutation in which the aspartic acid at position 65 is substituted with proline, a mutation in which the asparagine at position 66 is substituted with glycine, a mutation in which the threonine at position 101 is substituted with proline, and a mutation in which the serine at position 102 is substituted with asparagine, the xylanase variant comprising or consisting of the amino acid sequence of SEQ ID NO: 19 (the amino acid sequence of SEQ ID NO: 19 does not comprise the signal peptide region described above);

a xylanase variant derived from the amino acid sequence of SEQ ID NO: 1 comprising mutations in which the amino acids at both the positions of position 78 and position 155 are each substituted with alanine, and comprising a substitution of serine at position 35 with cysteine, a substitution of asparagine at position 44 with histidine, a substitution of tyrosine at position 61 with methionine, a substitution of threonine at position 62 with cysteine, a substitution of asparagine at position 63 with leucine, a substitution of aspartic acid at position 65 is substituted with proline, a substitution of asparagine at position 66 with glycine, a substitution of threonine at position 101 with proline, and a substitution of serine at position 102 with asparagine, the xylanase variant comprising or consisting of the amino acid sequence of SEQ ID NO: 20 (the amino acid sequence of SEQ ID NO: 20 does not comprise the signal peptide region described above).

The xylanase variant or a portion thereof also comprises a protein having an amino acid sequence wherein the amino acids substituted (if any) at the positions corresponding to position 78, position 80, position 117, position 155, position 169, and position 203 as well as position 35, position 44, position 61, position 62, position 63, position 65, position 66, position 101, and position 102 in the amino acid sequence of SEQ ID NO: 1 described above are not mutated, and wherein the amino acid sequence has a deletion, a substitution, an addition or an insertion of one or several amino acids, the protein having a xylanase activity. The range of “one or several” is the range defined above. In addition, the substitution of the amino acids other than the amino acids substituted (if any) at the positions corresponding to position 78, position 80, position 117, position 155, position 169, and position 203 as well as position 35, position 44, position 61, position 62, position 63, position 65, position 66, position 101, position 102 in the amino acid sequence of SEQ ID NO: 1 described above may be, for example, a conservative amino acid substitution. The “conservative amino acid substitution” is as defined above.

The xylanase variant or a portion thereof also comprises a protein comprising, more preferably consisting of an amino acid sequence wherein the amino acids substituted (if any) at the positions corresponding to position 78, position 80, position 117, position 155, position 169, and position 203 as well as position 35, position 44, position 61, position 62, position 63, position 65, position 66, position 101, and position 102 in the amino acid sequence of SEQ ID NO: 1 described above are not mutated, and wherein the amino acid sequence has an identity of preferably 90% or more, more preferably 91% or more, more preferably 92% or more, more preferably 93% or more, more preferably 94% or more, more preferably 95% or more, more preferably 96% or more, more preferably 97% or more, more preferably 98% or more, more preferably 99% or more, more preferably the same as or more than these, to the amino acid sequence of the xylanase variant or a portion thereof described above except the substituted amino acids, as calculated using a sequence analysis software such as BLAST (with, for example, default parameters, i.e., initial setting parameters), the protein having a xylanase activity. The “amino acid identity” is as defined above.

### (2) Method of Producing Xylanase Variant

The xylanase variant can be produced, for example, by preparing a DNA encoding the amino acid sequence of the xylanase variant or a portion thereof described above in (1), ligating the DNA to an expression vector, introducing the expression vector into a host, producing the xylanase variant as a heterologous or homologous protein, and isolating and purifying the xylanase variant. The codon usage for encoding the amino acid sequence may be the same as that for the filamentous fungus from which the xylanase is derived, for example, Acremonium cellulolyticus or Trichoderma reesei, or may be changed in accordance with the codon usage in the host.

As the method of preparing a DNA encoding a xylanase variant described above, a conventionally known technique can be used, and examples of such techniques include a method in which a DNA encoding an amino acid sequence of interest is totally synthesized by gene synthesis, or a method in which a mutation is introduced into a DNA encoding a xylanase or a portion thereof isolated from a filamentous fungus by site-directed mutagenesis so that the DNA encoding an amino acid at a specified position described above encodes a specified different amino acid and the like. The site-directed mutagenesis for inducing a mutation at a site of interest in a DNA can be performed using conventional, routinely used PCR. In other words, a DNA encoding a xylanase in which mutations are introduced at specified positions can be obtained by performing PCR using a filamentous fungus-derived gene as a template and using a primer pair for cloning the xylanase in which mutations are introduced at specified positions, wherein the primer pair is designed on the basis of a known base sequence encoding a filamentous fungus xylanase. As a “gene,” those selected from DNA, genome DNA, cDNA, RNA, mRNA, cDNA synthesized therefrom, DNA/RNA hybrids and the like can be used. When the base sequence encoding the subject filamentous fungus xylanase is not known, the subject filamentous fungus-derived xylanase gene can be obtained by: utilizing a primer pair designed on the basis of a known base sequence encoding another filamentous fungus xylanase and using the subject filamentous fungus gene as a template; or screening the gene pool of the subject filamentous fungus using a probe designed on the basis of a known base sequence encoding another filamentous fungus xylanase. The resulting xylanase gene can be used as a template for introduction of mutations. When a plurality of mutations are introduced, PCR is carried out using, as a template, a DNA into which one mutation has been introduced and using a primer pair for cloning the xylanase in which a mutation has been introduced at another specified position, whereby a DNA encoding a xylanase in which an additional mutation is introduced at the another specified position can be obtained. Preparation of a gene used as a template and PCR can be carried out in accordance with a known molecular biological technique (for example, a method described in Green, M. R. and Sambrook, J., 2012, Molecular Cloning: A Laboratory Manual Fourth Ed., Cold Spring Harbor Laboratory Press, Cold Spring Harbor, N.Y.).

Examples of DNAs encoding the xylanase variant include a DNA encoding a xylanase variant as described above in (1).

For example, as a DNA encoding the xylanase, a DNA encoding a xylanase isolated from Acremonium cellulolyticus can be utilized. Alternatively, a DNA that can be utilized as a DNA encoding a xylanase is: a DNA comprising or consisting of the base sequence shown in SEQ ID NO: 67; or a DNA comprising or consisting of the base sequence shown in SEQ ID NO: 68 obtained by removing the region encoding the signal peptide from the base sequence shown in SEQ ID NO: 67. These DNAs can be obtained by isolating a DNA from Acremonium cellulolyticus in accordance with a known method and carrying out DNA amplification using a technique such as PCR using a primer pair for cloning a xylanase designed on the basis of the base sequence encoding the xylanase from Acremonium cellulolyticus. By introducing mutations at specified positions in the obtained DNA, a DNA encoding the xylanase variant having an amino acid sequence or a portion thereof derived from the amino acid sequence of SEQ ID NO: 1 wherein the amino acids at one or more positions selected from position 78, position 80, position 117, position 155, position 169, and position 203 are each substituted, and having a xylanase activity can be obtained. More specific examples of such DNAs encoding the xylanase variant include a DNA comprising or consisting of the following DNAs:

a DNA encoding a xylanase variant derived from the amino acid sequence of SEQ ID NO: 1 comprising a mutation in which the aspartic acid at position 78 is substituted with alanine, the DNA comprising or consisting of the base sequence of SEQ ID NO: 69 (the DNA does not encode the signal peptide region described above);

a DNA encoding a xylanase variant derived from the amino acid sequence of SEQ ID NO: 1 comprising a mutation in which the threonine at position 80 is substituted with alanine, the DNA comprising or consisting of the base sequence of SEQ ID NO: 70 (the DNA does not encode the signal peptide region described above);

a DNA encoding a xylanase variant derived from the amino acid sequence of SEQ ID NO: 1 comprising a mutation in which the leucine at position 117 is substituted with asparagine, the DNA comprising or consisting of the base sequence of SEQ ID NO: 71 (the DNA does not encode the signal peptide region described above);

a DNA encoding a xylanase variant derived from the amino acid sequence of SEQ ID NO: 1 comprising a mutation in which the arginine at position 155 is substituted with alanine, the DNA comprising or consisting of the base sequence of SEQ ID NO: 72 (the DNA does not encode the signal peptide region described above);

a DNA encoding a xylanase variant derived from the amino acid sequence of SEQ ID NO: 1 comprising a mutation in which the glutamine at position 169 is substituted with alanine, the DNA comprising or consisting of the base sequence of SEQ ID NO: 73 (the DNA does not encode the signal peptide region described above);

a DNA encoding a xylanase variant derived from the amino acid sequence of SEQ ID NO: 1 comprising a mutation in which the asparagine at position 203 is substituted with tryptophan, the DNA comprising or consisting of the base sequence of SEQ ID NO: 74 (the DNA does not encode the signal peptide region described above);

a DNA encoding a xylanase variant derived from the amino acid sequence of SEQ ID NO: 1 comprising mutations in which the amino acids at both position 78 and position 155 are each substituted with alanine, the DNA comprising or consisting of the base sequence of SEQ ID NO: 75 (the DNA does not encode the signal peptide region described above).

In addition to those described above, a DNA encoding a xylanase isolated from Trichoderma reesei can be utilized as a DNA encoding the xylanase. Alternatively, a DNA that can be utilized as a DNA encoding a xylanase is a DNA comprising or consisting of the base sequence shown in SEQ ID NO: 91, or a DNA comprising or consisting of the base sequence shown in SEQ ID NO: 92 obtained by removing the region encoding the signal peptide from the base sequence shown in SEQ ID NO: 91. These DNAs can be obtained by isolating a DNA from Trichoderma reesei in accordance with a known method and carrying out DNA amplification using a technique such as PCR using a primer pair for cloning a xylanase designed on the basis of the base sequence encoding the xylanase from Trichoderma reesei. By introducing mutations at specified positions in the obtained DNA, a DNA encoding the xylanase variant having an amino acid sequence or a portion thereof derived from the amino acid sequence of SEQ ID NO: 1 wherein the amino acids at the positions corresponding to position 78 and/or position 80 are each substituted, and having a xylanase activity can be obtained. More specific examples of such DNAs encoding the xylanase variant include a DNA comprising or consisting of the following DNAs:

a DNA encoding a xylanase variant derived from the amino acid sequence of SEQ ID NO: 87 comprising a mutation in which the aspartic acid corresponding to position 78 in SEQ ID NO: 1 is substituted with alanine, the DNA comprising or consisting of the base sequence of SEQ ID NO: 93 (the DNA does not encode the signal peptide region described above);

a DNA encoding a xylanase variant derived from the amino acid sequence of SEQ ID NO: 87 comprising a mutation in which the valine corresponding to position 80 in SEQ ID NO: 1 is substituted with alanine, the DNA comprising or consisting of the base sequence of SEQ ID NO: 94 (the DNA does not encode the signal peptide region described above).

In another aspect, a DNA encoding the xylanase variant comprises or consists of a DNA encoding a polypeptide having an amino acid sequence or a portion thereof derived from the amino acid sequence of SEQ ID NO: 1 wherein the amino acids at one or more positions selected from position 78, position 80, position 117, position 155, position 169, and position 203 described above are substituted and, in addition, wherein the amino acids at position 35, position 44, position 61, position 62, position 63, position 65, position 66, position 101, and position 102 are substituted, the polypeptide having a xylanase activity.

Examples of such DNAs encoding the xylanase variant include the following:

a DNA encoding a xylanase variant derived from the amino acid sequence of SEQ ID NO: 1 comprising a mutation in which the aspartic acid at position 78 is substituted with alanine, and comprising a mutation in which the serine at position 35 is substituted with cysteine, a mutation in which the asparagine at position 44 is substituted with histidine, a mutation in which the tyrosine at position 61 is substituted with methionine, a mutation in which the threonine at position 62 is substituted with cysteine, a mutation in which the asparagine at position 63 is substituted with leucine, a mutation in which the aspartic acid at position 65 is substituted with proline, a mutation in which the asparagine at position 66 is substituted with glycine, a mutation in which the threonine at position 101 is substituted with proline, and a mutation in which the serine at position 102 is substituted with asparagine, the DNA comprising or consisting of the base sequence of SEQ ID NO: 84 (the base sequence of SEQ ID NO: 84 does not comprise the region encoding the signal peptide described above);

a DNA encoding a xylanase variant derived from the amino acid sequence of SEQ ID NO: 1 comprising a mutation in which the threonine at position 80 is substituted with alanine, and comprising a mutation in which the serine at position 35 is substituted with cysteine, a mutation in which the asparagine at position 44 is substituted with histidine, a mutation in which the tyrosine at position 61 is substituted with methionine, a mutation in which the threonine at position 62 is substituted with cysteine, a mutation in which the asparagine at position 63 is substituted with leucine, a mutation in which the aspartic acid at position 65 is substituted with proline, a mutation in which the asparagine at position 66 is substituted with glycine, a mutation in which the threonine at position 101 is substituted with proline, and a mutation in which the serine at position 102 is substituted with asparagine, the DNA comprising or consisting of the base sequence of SEQ ID NO: 85 (the base sequence of SEQ ID NO: 85 does not comprise the region encoding a signal peptide described above);

a DNA encoding a xylanase variant derived from the amino acid sequence of SEQ ID NO: 1 comprising mutations in which amino acids of both position 78 and position 155 are each substituted with alanine, and comprising a mutation in which the serine at position 35 is substituted with cysteine, a mutation in which the asparagine at position 44 is substituted with histidine, a mutation in which the tyrosine at position 61 is substituted with methionine, a mutation in which the threonine at position 62 is substituted with cysteine, a mutation in which the asparagine at position 63 is substituted with leucine, a mutation in which the aspartic acid at position 65 is substituted with proline, a mutation in which the asparagine at position 66 is substituted with glycine, a mutation in which the threonine at position 101 is substituted with proline, and a mutation in which the serine at position 102 is substituted with asparagine, the DNA comprising or consisting of the base sequence of SEQ ID NO: 86 (the base sequence of SEQ ID NO: 86 does not comprise the region encoding a signal peptide described above).

DNAs encoding the xylanase variant include a DNA comprising or consisting of the following base sequence, as long as the amino acids substituted (if any) in the amino acid sequence of the xylanase variant or a portion thereof described above at the positions corresponding to position 78, position 80, position 117, position 155, position 169, and position 203 as well as position 35, position 44, position 61, position 62, position 63, position 65, position 66, position 101, and position 102 in the amino acid sequence of SEQ ID NO: 1 described above are not mutated, and as long as the DNA encodes a polypeptide having a xylanase activity:

a base sequence derived from a base sequence of a DNA encoding the xylanase variant described above, having a deletion, a substitution, an addition, or an insertion of one to multiple bases; for example, a base sequence derived from a base sequence shown in SEQ ID NO: 1 having a deletion, a substitution, an addition, or an insertion of 1 to 100 bases, preferably 1 to 50 bases, more preferably 1 to 10 bases (the base substitution may be a substitution producing a conservative amino acid substitution);

a base sequence having a base identity of 80% or more, more preferably 85% or more, more preferably 90% or more, more preferably 91% or more, more preferably 92% or more, more preferably 93% or more, more preferably 94% or more, still more preferably 95% or more, still more preferably 96% or more, still more preferably 97% or more, still more preferably 98% or more, most preferably 99% or more, to the base sequence of the DNA encoding the xylanase variant described above; comparison of base sequences can be performed by a known technique using, for example, BLAST and the like with, for example, default setting;

a base sequence hybridizing with a DNA consisting of a sequence complementary with the base sequence described above under stringent conditions. The “stringent conditions” refer to conditions under which a specific hybrid is formed and no nonspecific hybrid is formed, for example, conditions under which hybridization is performed at 42 to 55° C. in a solution comprising 2 to 6×SSC (the composition of 1×SSC: 0.15 M NaCl, 0.015 M sodium citrate, pH 7.0) and 0.1 to 0.5% SDS, and washing is performed at 55 to 65° C. in a solution comprising 0.1 to 0.2×SSC and 0.1 to 0.5% SDS.

The DNA encoding the xylanase variant prepared as described above is ligated to the downstream of a promoter in a suitable expression vector using a restriction enzyme and a DNA ligase, whereby an expression vector comprising the DNA can be produced.

Examples of expression vectors include bacterial plasmids, yeast plasmids, phage DNAs (such as lambda phage), retroviruses, baculoviruses, vaccinia viruses, virus DNA of adenoviruses and the like, SV40 derivatives and the like, Agrobacterium vectors for plant cells and the like, and any other vector can be used as long as the vector is replicable and viable in a host cell. Examples when the host is E. coli include pUC, pET, pBAD and the like. In addition, examples when the host is a yeast include pPink-HC, pPink-LC, pPinkα-HC, pPCIZ, pPCIZα, pPCI6, pPCI6α, pFLD1, pFLD1α, pGAPZ, pGAPZα, pPIC9K, pPIC9, pD912, pD915 and the like.

The promoter may be any promoters suitable for a host used for expression of genes. Examples of promoters when the host is E. Coli include lac promoters, Trp promoters, PL promoters, PR promoters and the like, and examples of promoters when the host is a yeast include AOX1 promoters, TEF1 promoters, ADE2 promoters, CYC1 promoters, GAL-L1 promoters, GAP promoters and the like.

Preferable examples of host cells include E. coli, bacterial cells, yeast cells, fungal cells, insect cells, plant cells, animal cells and the like. Examples of yeast cells include Pichia, Saccharomyces, Schizosaccharomyces and the like. Examples of fungal cells include Aspergillus, Trichoderma and the like. Examples of insect cells include Sf9 and the like, examples of plant cells include dicots and the like, and examples of animal cells include CHO, HeLa, HEK293 and the like. A host is preferably a eukaryotic microorganism, more preferably a yeast cell or a fungal cell. Yeast cells and fungal cells used as hosts can have advantages in that the amount of production of enzymes is high, that they can produce and secretes enzymes extracellularly, and/or that the heat resistance of enzymes can be enhanced.

Transformation or transfection can be carried out by a known method such as a calcium phosphate method or an electroporation method. The xylanase variant can be obtained by collecting a product expressed under the control of a promoter in a host cell transformed or transfected as described above. In the expression, the transformed or transfected host cell is proliferated or grown to a suitable cell density, the promoter is then induced by a chemical induction means such as temperature shift or isopropyl-1-thio-β-D-galactoside (IPTG) addition, and the cells are further cultured for a certain period of time. Alternatively, the promoter can be induced by a sugar comprised in the culture medium, and culture of the cell and expression can be performed simultaneously.

The xylanase variant is purified directly from the culture medium when the xylanase variant of interest is exported extracellularly, or purified after breaking the cell using a physical means such as ultrasonic breaking or mechanical breaking or a chemical means such as a cell solubilizer when the xylanase variant is present intracellularly. Specifically, the xylanase variant can be purified partially or completely from a culture medium of recombinant cells by combining techniques such as ammonium sulfate or ethanol precipitation, acid extraction, negative-ion- or positive-ion-exchange chromatography, reversed phase high performance liquid chromatography, affinity chromatography, gel filtration chromatography, electrophoresis and the like.

### (3) Enzyme Composition for Decomposing Biomass Comprising Xylanase Variant

The biomass enzyme composition is an enzyme composition used for applications in decomposing biomass, comprising at least the xylanase variant as an active constituent for hydrolysis of biomass. Biomass refers to biological plant body, and examples thereof include herbaceous plants, woody plants, algae, seaweeds, sugar production crops, resources crops, cereals and the like. These biomasses each comprise disaccharides or higher polysaccharides, and the enzyme composition for decomposing biomass can hydrolyze the polysaccharides. Cellulose-containing biomass can be particularly preferably used. Cellulose-containing biomass is biological resources containing cellulose components. Specifically, cellulose-containing biomass refers to: herbaceous biomass such as bagasse, switchgrass, napier grass, erianthus, corn stover, corn hull, rice straw, wheat straw, and wheat bran; woody biomass such as trees and waste construction materials; and biomass derived from the aquatic environment such as algae and sea grasses. Such cellulose-based biomass comprises an aromatic polymer such as lignin in addition to cellulose and hemicellulose (hereinafter, cellulose and hemicellulose are collectively referred to as “cellulose”).

The xylanase variant comprised in the enzyme composition for decomposing biomass can be used, whether it is purified or crude. In addition, the xylanase variant comprised in the enzyme composition for decomposing biomass may be immobilized on a solid phase. Examples of solid phases include polyacrylamide gels, polystyrene resins, porous glass, metal oxides and the like (but are not particularly limited to these). The xylanase variant immobilized on a solid phase is advantageous in that the xylanase variant can be used continuously and repetitively. Furthermore, processed materials of cells transformed with the DNA encoding the xylanase variant described above can also be utilized as a crude xylanase variant. Examples of the “processed materials of cells” include: transformed cells immobilized on a solid phase; killed microbes and crushed materials of the transformed cells, and those immobilized on a solid phase and the like.

The enzyme composition for decomposing biomass may comprise other enzymes in addition to the xylanase variant. The enzyme composition preferably comprises a hydrolase related to biomass decomposition. Examples of such other enzymes include cellobiohydrolase, endoglucanase, β-glucosidase, β-xylosidase, mannanase, mannosidase, glucoamylase, α-amylase, esterase, lipase and the like.

These other enzymes are preferably enzymes produced by microorganisms such as filamentous fungi. Examples of filamentous fungi include microorganisms such as Trichoderma, Aspergillus, Cellulomonas, Clostridium, Streptomyces, Humicola, Acremonium, Irpex, Mucor, Talaromyces and the like. These microorganisms produce an enzyme in a culture medium, and the culture medium may be used as it is as an unpurified enzyme to be combined with the xylanase variant and thereby formed into the enzyme composition, or the culture medium may be purified and formulated to be combined with the xylanase variant and thereby formed into the enzyme composition.

A filamentous fungus for producing the other enzymes described above is preferably a filamentous fungus derived from Trichoderma. Among Trichoderma, a cellulase mixture derived from Trichoderma reesei can be more preferably used. Examples of cellulase mixtures derived from Trichoderma reesei include cellulase mixtures derived from Trichoderma reesei QM9414 (T. reesei QM9414), Trichoderma reesei QM9123 (T. reesei QM9123), Trichoderma reesei RutC-30 (T. reesei Rut-30), Trichoderma reesei PC3-7 (T. reesei PC3-7), Trichoderma reesei CL-847 (T. reesei CL-847), Trichoderma reesei MCG77 (T. reesei MCG77), Trichoderma reesei MCG80 (T. reesei MCG80), and Trichoderma viride QM9123 (T. viride QM9123). Furthermore, the filamentous fungus may be a variant strain derived from the Trichoderma having a cellulase productivity improved by mutagenesis using a mutagenesis agent, ultraviolet irradiation or the like.

In addition, substances other than enzymes, for example, protease inhibitors, dispersing agents, dissolution promoters, stabilizing agents, buffering agents, antiseptic agents and the like, may be added to the enzyme composition for decomposing biomass.

The enzyme composition for decomposing biomass can be added to biomass to be used in a method of producing a sugar solution. The enzyme composition for decomposing biomass can be used in production of xylooligosaccharide, particularly xylotriose. As used herein, a sugar solution refers to a solution at least comprising saccharides derived from hydrolysis of biomass-derived polysaccharides into lower molecular weight sugars. Examples of sugar components in a sugar solution include xylose, glucose, cellobiose, xylobiose, xylotriose, xylotetraose, xylopentaose, mannose, arabinose, sucrose, fructose and the like. The enzyme composition for decomposing biomass comprises at least a xylanase variant and, accordingly, a sugar solution obtained using the enzyme composition often comprises xylose, xylobiose, xylotriose, xylotetraose, xylopentaose, and xylohexaose. The composition of a sugar solution can be analyzed by high performance liquid chromatography and the like. A biomass used in production of a sugar solution may be any one of the biomasses described above, and is preferably pre-treated to be used for the purpose of increasing the sugar yield from the biomass. The pre-treatment refers to partially decomposing lignin and hemicellulose in biomass in advance using acid, alkali, hot water under pressure and the like. In a method of producing a sugar solution, it is preferable to add the enzyme composition for decomposing biomass to biomass, and allow the reaction to take place for one minute to 240 hours under the conditions: a temperature of 40° C. to 100° C., a treatment pH of 3 to 7, and a biomass concentration of 0.1 to 30%. Using this range makes it possible to maximize the decomposition efficiency of the enzyme composition for decomposing biomass.

The enzyme composition for decomposing biomass used in a method of producing a sugar solution can be collected and further reused. The xylanase variant comprised in the collected enzyme composition for decomposing biomass can retain an activity of 50% or more, 60% or more, 70% or more, or 80% or more, preferably 90% or more, of the activity before being used in the method of producing a sugar solution. In addition, the higher these values are, the higher the enzyme collecting performance is considered to be. The enzyme collecting performance exhibits a particularly high value when the enzyme composition for decomposing biomass is a xylanase variant having an amino acid sequence derived from the amino acid sequence of SEQ ID NO: 1 wherein the amino acids at one or more positions selected from amino acid position 78, position 80, position 117, position 155, position 169, and position 203 are substituted, and in addition, wherein the amino acids at position 35, position 44, position 61, position 62, position 63, position 65, position 66, position 101, and position 102 are substituted. The enzyme composition for decomposing biomass can be collected by the following technique. The enzyme composition for decomposing biomass is added to biomass to perform hydrolysis reaction, and then the hydrolysate is allowed to undergo solid-liquid separation. The solution components obtained by solid-liquid separation comprise the enzyme composition for decomposing biomass and sugar components, and the enzyme composition for decomposing biomass described above and the sugar components are separated by filtration using an ultrafiltration membrane. In using an ultrafiltration membrane to separate the enzyme composition for decomposing biomass and the sugar components, the molecular weight cutoff of the ultrafiltration membrane is not limited as long as the molecular weight cutoff allows monosaccharides and oligosaccharides (from disaccharides to decasaccharides) to permeate the ultrafiltration membrane and can block the enzyme composition for decomposing biomass. Specifically, the molecular weight cutoff may be in the range of 2,000 to 50,000, more preferably in the molecular weight cutoff range of 5,000 to 50,000, still more preferably in the molecular weight cutoff range of 10,000 to 30,000, considering the separation of the enzyme from the contaminants exhibiting an action of blocking the enzymatic reaction. Examples of raw materials that can be used for ultrafiltration membranes include polyethersulfone (PES), polysulfone (PS), polyacrylonitrile (PAN), polyvinylidene fluoride (PVDF), regenerated cellulose, cellulose, cellulose ester, sulfonated polysulfone, sulfonated polyethersulfone, polyolefin, polyvinyl alcohol, polymethyl methacrylate, polytetrafluoroethylene and the like, and ultrafiltration membranes the raw material of which is a synthetic polymer such as PES or PVDF are preferably used. In the collection and/or reuse of the enzyme composition for decomposing biomass, the xylanase variant comprised in the enzyme composition for decomposing biomass is preferably a xylanase variant having an amino acid sequence derived from the amino acid sequence of SEQ ID NO: 1 wherein the amino acids at 11 positions: position 35, position 44, position 62, position 63, position 101, position 102, position 61, position 65, position 66, position 78, and position 155 are substituted, and is more preferably a xylanase variant having the amino acid sequence of SEQ ID NO: 20.

A sugar solution obtained by the method of producing a sugar solution comprises monosaccharide components such as glucose and xylose and, accordingly, can be used as a raw sugar for ethanol, lactic acid and the like. In addition, a sugar solution obtained by the method of producing a sugar solution comprises xylooligosaccharide, xylobiose, xylotriose and the like, and accordingly can be used as an oligosaccharide in prebiotics applications, and used as human health food and livestock feed.

### EXAMPLES

Below, our variants and compositions will be specifically described with reference to Examples. However, this disclosure is not to be limited to these Examples.

### Reference Example 1: Measurement of Protein Concentration

The protein concentrations of a xylanase and a xylanase variant were measured by the BCA method. A solution in an amount of 25 μL comprising the xylanase or the xylanase variant was mixed with 200 μL of a BCA reagent, and the resulting mixture was allowed to react at 37° C. for 30 minutes and thereby develop colors. The protein concentrations were determined by colorimetry with the absorbances measured at 570 nm using bovine serum albumin as a standard material.

### Reference Example 2: Preparation of Trichoderma-Derived Cellulase

Trichoderma-derived cellulase was prepared by the following method.

### Reference Example 3: Measurement of Sugar Concentration

Xylotriose and xylose were subjected to quantitative analysis based on a calibration curve prepared from standard samples of xylotriose and xylose using a Hitachi high performance liquid chromatograph, LaChrom Eite (Hitachi, Ltd.). The analysis conditions are as follows:

Column: KS802, KS803 (Shodex)

Mobile Phase: water

Detection Method: RI

Flow Rate: 0.5 mL/min

Temperature: 75° C.

### Preculture

The following materials were added to distilled water to obtain 5% (w/vol) corn steep liquor, 2% (w/vol) glucose, 0.37% (w/vol) ammonium tartrate, 0.14% (w/vol) ammonium sulfate, 0.2% (w/vol) potassium dihydrogen phosphate, 0.03% (w/vol) calcium chloride dihydrate, 0.03% (w/vol) magnesium sulfate heptahydrate, 0.02% (w/vol) zinc chloride, 0.01% (w/vol) ferric chloride (III) hexahydrate, 0.004% (w/vol) copper sulfate (II) pentahydrate, 0.0008% (w/vol) manganese chloride tetrahydrate, 0.0006% (w/vol) boric acid, and 0.0026% (w/vol) hexaammonium heptamolybdate tetrahydrate, and 100 ml of the resulting mixture was placed in a 500 ml Er-lenmeyer flask with baffles, and autoclaved at 121° C. for 15 minutes. The resulting mixture was left to cool down, and then, to the mixture, PE-M and Tween 80 that had each been autoclaved at 121° C. for 15 minutes separately from the mixture were added at 0.01% (w/vol) each. Into this preculture medium, Trichoderma reesei ATCC66589 (distributed by ATCC) was inoculated at 1×10⁵/mL, and cultured with shaking at 28° C. at 180 rpm for 72 hours as a preculture (using a shaking device, BIO-SHAKER BR-40LF, manufactured by TAITEC Corporation).

### Main Culture

The following materials were added to distilled water to obtain 5% (w/vol) corn steep liquor, 2% (w/vol) glucose, 10% (w/vol) cellulose (Avicel), 0.37% (w/vol) ammonium tartrate, 0.14% (w/vol) ammonium sulfate, 0.2% (w/vol) potassium dihydrogen phosphate, 0.03% (w/vol) calcium chloride dihydrate, 0.03% (w/vol) magnesium sulfate heptahydrate, 0.02% (w/vol) zinc chloride, 0.01% (w/vol) ferric chloride (III) hexahydrate, 0.004% (w/vol) copper sulfate (II) pentahydrate, 0.0008% (w/vol) manganese chloride tetrahydrate, 0.0006% (w/vol) boric acid, and 0.0026% (w/vol) hexaammonium heptamolybdate tetrahydrate, and 2.5 L of the resulting mixture was placed in a 5 L capacity stirring jar (DPC-2A, manufactured by ABLE Corporation), and autoclaved at 121° C. for 15 minutes. The resulting mixture was left to cool down, and then, to the mixture, PE-M and Tween 80 that had each been autoclaved at 121° C. for 15 minutes separately from the mixture were added at 0.1% each, and into the resulting mixture, 250 mL of Trichoderma reesei PC3-7 precultured in a liquid culture medium in advance by the method described above was inoculated. Then culturing was performed at 28° C. at 300 rpm at an aeration rate of 1 vvm for 87 hours, followed by centrifugation, and then the supernatant was filtered through a membrane (Stericup-GV, made of PVDF, manufactured by Millipore). To this culture solution prepared under the conditions described above, β-glucosidase (Novozyme 188) was added at a protein weight ratio of 1/100, and the resulting mixture was used as Trichoderma-derived cellulase in the Examples below.

### Comparative Example 1: Cloning of Wild-Type Xylanase (Having the Amino Acid Sequence of SEQ ID NO: 1) Isolated from Acremonium cellulolyticus

A DNA encoding a wild-type xylanase having the amino acid sequence of SEQ ID NO: 1 was isolated from Acremonium cellulolyticus CF strains by RT-PCR, and a DNA that encodes a protein obtained by removing the signal peptide region from the wild-type xylanase and has the base sequence of SEQ ID NO: 68 was cloned at the NdeI site and BamHI site of pET11a (Novagen). “ATG” in the base sequence CATATG at the NdeI site of pET11a was used as a methionine codon and as the translation initiation site, and the 3′ end comprised the stop codon “TAG.”

The pET11a comprising the base sequence of SEQ ID NO: 68 was cloned in BL21(DE3) strains (from Novagen). The resulting recombinant BL21(DE3) strains were cultured in an LB culture medium comprising 100 mg/L of ampicillin sodium at 37° C. until the OD600 value became 0.6, and then, to the resulting culture, 200 μM isopropyl-β-D-1-thiogalactopyranoside (IPTG) was added, and expression of a xylanase having the amino acid sequence of SEQ ID NO: 2 was induced. The induction of expression was carried out with the culture medium temperature maintained at 16° C. for 20 hours, and then, recombinant BL21(DE3) strains were harvested by centrifugation at 4° C. at 5,000×g for 15 minutes. The harvested fungus bodies were re-suspended in a Tris buffer pH 8 (20 mM Tris HCl, 50 mM NaCl). The buffer comprising fungus bodies was subjected to three cycles in total of complete freezing at −80° C. for one hour followed by thawing at room temperature, whereby the soluble protein in the fungus bodies was extracted into the buffer. Then, the buffer was centrifuged at 18,000 rpm at 4° C. for 20 minutes to separate into the supernatant and the fungus body residue. The supernatant was passed through a Q-HP column (from GE) that had been equilibrated in advance using a Tris buffer (20 mM, pH 8), and the xylanase of interest was allowed to be adsorbed by the column, and then eluted with a NaCl concentration gradient. As a xylanase fraction, a solution obtained at a NaCl concentration of 200 to 400 mM was collected. Then, the xylanase fraction was further dialyzed with a Tris buffer (20 mM, pH 8, 2 M NaCl), and was passed through a Butyl HP column (GE), which adsorbed the xylanase. The xylanase was eluted with a NaCl concentration gradient, and the fraction eluted with 1 M NaCl was collected. The fraction was further passed through a Superdex 200 16/60 gel filtration column (GE) for purification. The resulting purified xylanase was examined for impurities using SDS-PAGE.

### Example 1: Preparation of DNA Encoding Xylanase Variant Comprising Substitution at Any One of Position 78, Position 80, Position 117, Position 155, Position 169, and Position 203, and Recombinant Expression with E. Coli

In the Example, a DNA encoding a xylanase variant comprising a substitution at any one of position 78, position 80, position 117, position 155, position 169, and position 203 was prepared by the following procedures.

Inverse PCR was carried out using, as a template, pET11a (Comparative Example 1) comprising the base sequence (SEQ ID NO: 68) encoding the amino acid sequence (SEQ ID NO: 2) obtained by removing the signal peptide consisting of 34 amino acid residues at the N-terminus from the amino acid sequence (SEQ ID NO: 1) of the wild-type xylanase of Acremonium cellulolyticus, and using a primer pair (Fw: forward primer, Rv: reverse primer) shown in Table 1. For the PCR, PrimeSTAR Max (from Takara Bio Inc.) was used. A DNA encoding a xylanase variant having amino acid substitutions at specified positions was prepared by transforming DH5a strains (Novagen) using a reaction solution comprising PCR-amplified fragments. The primer pairs used are shown in Table 1 (SEQ ID NO: 21 to SEQ ID NO: 32). In addition, the base sequences of the obtained DNAs encoding a xylanase variant (having the initiating site at the amino acid of position 35) are shown as SEQ ID NO: 69, SEQ ID NO: 70, SEQ ID NO: 71, SEQ ID NO: 72, SEQ ID NO: 73, and SEQ ID NO: 74 (Table 1). In addition, the amino acid sequences of the respective xylanase variants are shown in SEQ ID NO: 3, SEQ ID NO: 4, SEQ ID NO: 5, SEQ ID NO: 6, SEQ ID NO: 7, and SEQ ID NO: 8. The obtained DNA encoding a xylanase variant was cloned into the BL21 (DE3) strain, and expression of the xylanase variant was induced in accordance with the procedures for Comparative Example 1. Then, the recombinant BL21 (DE3) strain was harvested by centrifugation. The harvested fungus bodies were re-suspended in a Tris buffer pH 8 (20 mM Tris HCl, 50 mM NaCl), followed by three cycles of freezing and thawing to extract a crude enzyme solution. The crude enzyme solution was loaded into a HiTrapQ column (from GE), the protein was eluted with 0 to 1 M NaCl, and fractions showing a xylanase activity were collected. The collected fraction was dialyzed with 20 mM Tris HCl (pH 8.0) and loaded into a RESOURCE Q column (GE), and the xylanase variant was eluted with 0 to 0.25 M NaCl for purification. When the purity of the obtained fraction is low, the fraction was subjected to a HiPrep 200 μg. column (GE) that had been equilibrated in advance with a Tris buffer (20 mM Tris HCl, 50 mM NaCl), and thus, a purified enzyme was obtained.

TABLE 1
Primer Pairs for Introducing Mutators and Corresponding Xylanase Variants
(Amino Acid Sequence and Base Sequence) Used in Example 1
The underlines indicate mutation sites.

| | Amino Acid | Amino Acid | | | | Xylanase Variant | |
|---|---|---|---|---|---|---|---|
| | Before | After | | Primer Base Sequence | | Amino Acid | Base |
| Position | Substitution | Substitution | Fw/Rv | (5′→3′) | | Sequence | Sequence |
| Position | Aspartic Acid | Alanine | Fw | TTGCGGTGCGTTTAC | SEQ ID | SEQ ID | SEO ID |
| 78 | | | | ATCTGGCAAGGGCT | NO: 21 | NO: 3 | NO: 69 |
| | | | Rv | GATGTAAACGCACCG | SEQ ID | | |
| | | | | CAATTGACCCAGG | NO: 22 | | |
| Position | Threonine | Alanine | Fw | GTGACTTTGCGTCTG | SEQ ID | SEQ ID | SEO ID |
| 80 | | | | GCAAGGGCTGGAATC | NO: 23 | NO: 4 | NO: 70 |
| | | | Rv | TTGCCAGACGCAAAG | SEQ ID | | |
| | | | | TCACCGCAATTGAC | NO: 24 | | |
| Position | Leucine | Asparagine | Fw | GATCCTAACGTCGAA | SEQ ID | SEQ ID | SEO ID |
| 1*7 | | | | TACTACATCCTGGA | NO: 25 | NO: 5 | NO: 71 |
| | | | Rv | TTCGACGTTAGGATC | SEQ ID | | |
| | | | | AGTTGTCCAACCG | NO: 26 | | |
| Position | Arginine | Alanine | Fw | CAACACAGGCGGTCG | SEQ ID | SEQ ID | SEO ID |
| 155 | | | | AGGAACCCTCCATC | NO: 27 | NO: 6 | NO: 72 |
| | | | Rv | GGTCGACCGCCTGTG | SEQ ID | | |
| | | | | TTGAGTAGATATCG | NO: 28 | | |
| Position | Glutamine | Alanine | Fw | CTTCAATGCGTACTG | SEQ ID | SEQ ID | SEO ID |
| 169 | | | | GTCGGTTCGCAC | NO: 29 | NO: 7 | NO: 73 |
| | | | Rv | ACCAGTACGCATTGA | SEQ ID | | |
| | | | | AGGTGGAAGTTCC | NO: 30 | | |
| Position | Asparagine | Tryptophan | Fw | GTACTTATTGGTATA | SEQ ID | SEQ ID | SEO ID |
| 203 | | | | TGATTGTGTCTAGA | NO: 31 | NO: 8 | NO: 74 |
| | | | Rv | ATCATATACCAATAA | SEQ ID | | |
| | | | | GTACCCATTTCAAG | NO: 32 | | |

### Example 2: Preparation of DNA Encoding Xylanase Variant Comprising Two Substitutions at Position 78 and Position 155, and Recombinant Expression with E. Coli

With pET11a comprising a DNA that was prepared in Example 1 and that encodes the xylanase variant (SEQ ID NO: 3) obtained by introducing a mutation at position 78, a primer pair (SEQ ID NO: 27 and SEQ ID NO: 28) for introducing a mutation at position 155 was further used to prepare a DNA (SEQ ID NO: 75) encoding a xylanase variant having two substitutions with alanine each at position 78 and position 155. The primer pairs used for introducing the mutation are the two species shown in Table 2. The base sequence of the prepared DNA encoding a xylanase variant is shown in SEQ ID NO: 9. The obtained DNA encoding a xylanase variant was cloned in pET11a in accordance with the procedures for Example 1. Then, using pET11a comprising the DNA encoding a xylanase variant, expression and purification of the protein was performed in accordance with the procedures for Comparative Example 1 to obtain the xylanase variant (SEQ ID NO: 9) in Example 2.

TABLE 2
Primer Pairs for Introducing Mutations and Corresponding Xylanase Variants
(Amino Acid Sequence and Base Sequence) Used in Example 2
The underlines indicate mutation sites.

| | Amino Acid | Amino Acid | | | | Xylanase Variant | |
|---|---|---|---|---|---|---|---|
| | Before | After | Fw/ | | | Amino Acid | Base |
| Position | Substitution | Substitution | Rv | Primer Base Sequence (5′-3′) | | Sequence | Sequence |
| Position | Aspartic | Alanine | Fw | TTGCGGTGCGTTTACATCTGGCAAGGGCT | SEQ ID NO: 21 | SEQ ID NO: | SEQ ID NO: |
| 78 | Acid | | Rv | GATGTAAACGCACCGCAATTGACCCAGG | SEQ ID NO: 22 | 9 | 75 |
| Position | Arginine | Alanine | Fw | CAACACAGGCGGTCGACCAACCCTCCATC | SEQ ID NO: 27 | | |
| 155 | | | Rv | GGTCGACCGCCTGTGTTGAGTAGATATCG | SEQ ID NO: 28 | | |

### Comparative Example 2: Preparation of DNA Encoding Xylanase Variant Comprising Substitution at Any One of Position 48, Position 112, Position 121, Position 123, Position 128, Position 167, Position 171, and Position 212, and Recombinant Expression with E. Coli

As a Comparative Example, a xylanase variant comprising a substitution at any one of position 48, position 112, position 121, position 123, position 128, position 167, position 171, and position 212 was prepared. DNA encoding each xylanase variant was prepared by PCR using, as a template, pET11a comprising the base sequence of the xylanase of SEQ ID NO: 2, and using a primer pair (Fw: forward primer, Rv: reverse primer) shown in Table 3. For the PCR, PrimeSTAR Max (from Takara Bio Inc.) was used. The base sequences of the primer pairs used and the obtained DNAs encoding a xylanase variant are shown in SEQ ID NO: 76, SEQ ID NO: 77, SEQ ID NO: 78, SEQ ID NO: 79, SEQ ID NO: 80, SEQ ID NO: 81, SEQ ID NO: 82, and SEQ ID NO: 83 (Table 3). In addition, the amino acid sequences of the respective xylanase variants are shown in SEQ ID NO: 10, SEQ ID NO: 11, SEQ ID NO: 12, SEQ ID NO: 13, SEQ ID NO: 14, SEQ ID NO: 15, SEQ ID NO: 16, and SEQ ID NO: 17 (excluding the initiator methionine). The obtained DNA encoding a xylanase variant was cloned in pET11a in accordance with the procedures for Example 1. Then, using pET1a comprising the DNA encoding a xylanase variant, the expression and purification of the protein was performed in accordance with the procedures for Example 1 to obtain the variant in Comparative Example 2.

TABLE 3
Primer Pairs for Introducing Mutations and Corresponding Variants
(Amino Acid Sequence and Base Sequence) Used in Comparative Example 2
The underlines indicate mutation sites.

| | Amino Acid | Amino Acid | | | | Xylanase Variant | |
|---|---|---|---|---|---|---|---|
| | Before | After | Fw/ | | | Amino Acid | Base |
| Position | Substitution | Substitution | Rv | Primer Base Sequence (5′-3′) | | Sequence | Sequence |
| Position | Tyrosine | Alanine | Fw | CTACTACGCGTCGTTCTGGACCAACGGC | SEQ ID NO: 33 | SEQ ID NO: 10 | SEQ ID |
| 48 | | | Rv | CAGAACGACGCGTAGTAGCCGTTGTGCG | SEQ ID NO: 34 | | NO: 76 |
| Position | Tryptophan | Alanine | Fw | GTTTACGGTGCGACAACTGATCCTCTTGTC | SEQ ID NO: 35 | SEQ ID NO: 11 | SEQ ID |
| 112 | | | Rv | CAGTTGTCGCACCGTAAACGGCGAGATAAG | SEQ ID NO: 36 | | NO: 77 |
| Position | Tyrosine | Alanine | Fw | CGAATACGCGATCCTGGAGTCCTACGGTA | SEQ ID NO: 37 | SEQ ID NO: 12 | SEQ ID |
| 121 | | | Rv | CTCCAGGATCGCGTATTCGACAAGAGGAT | SEQ ID NO: 38 | | NO: 78 |
| Position | Leucine | Tyrosine | Fw | ATACTACATCTATGAGTCCTACGGTACAAT | SEQ ID NO: 39 | SEQ ID NO: 13 | SEQ ID |
| 123 | | | Rv | GTAGGACTCATAGATGTAGTATTCGACAAG | SEQ ID NO: 40 | | NO: 79 |
| Position | Threonine | Tyrosine | Fw | CTACGGTTACTATAACCCATCATCTGGC | SEQ ID NO: 41 | SEQ ID NO: 14 | SEQ ID |
| 128 | | | Rv | GGGTTATAGTAACCGTAGGACTCCAGGA | SEQ ID NO: 42 | | NO: 80 |
| Position | Phenylalanine | Alanine | Fw | CTTCCACCGCGAATCAGTACTGGTCGGTT | SEQ ID NO: 43 | SEQ ID NO: 15 | SEQ ID |
| 167 | | | Rv | GTACTGATTCGCGGTGGAAGTTCCCTCG | SEQ ID NO: 44 | | NO: 81 |
| Position | Tryptophan | Alanine | Fw | ATCAGTACGCGTCGGTTCGCACAGAGAAG | SEQ ID NO: 45 | SEQ ID NO: 16 | SEQ ID |
| 171 | | | Rv | CGAACCGACGCGTACTGATTGAAGGTGGA | SEQ ID NO: 46 | | NO: 82 |
| Position | Tyrosine | Alanine | Fw | CAGAAGGCGCGGAGAGCAGTGGTTCTAGT | SEQ ID NO: 47 | SEQ ID NO: 17 | SEQ ID |
| 212 | | | Rv | TCCTCTCCCCGCCTTCTCTACACACAATC | SEQ ID NO: 48 | | NO: 83 |

### Example 3: Preparation of DNA Encoding Xylanase Variant Comprising Substitution at Position 78, or Substitution at Position 80, or Substitutions at Positions 78 and 155, as Well as Substitutions at Position 35, Position 44, Position 62, Position 63, Position 101, Position 102, Position 61, Position 65, and Position 66, and Recombinant Expression with E. Coli

Primer pairs for further introducing mutations at position 35, position 44, position 62, position 63, position 101, position 102, position 61, position 65, and position 66 were used for pET11 comprising the DNA (SEQ ID NO: 69) encoding the xylanase variant having a substitution with alanine at position 78; pET11 comprising the DNA (SEQ ID NO: 70) encoding the xylanase variant having a substitution with alanine at position 80; and pET11 comprising the DNA (SEQ ID NO: 75) encoding the xylanase variant having a substitution with alanine at each of two positions, position 78 and position 155, prepared in Example 1 and Example 2, to prepare DNAs encoding a xylanase variant having a substitution at each position. The primer pairs used for introducing the mutations are the nine species shown in Table 4. The base sequences of the prepared DNAs encoding a xylanase variant are shown in SEQ ID NO: 84, SEQ ID NO: 85, and SEQ ID NO: 86. In addition, the amino acid sequences of the xylanase variants are shown in SEQ ID NO: 18, SEQ ID NO: 19, and SEQ ID NO: 20. The obtained DNA encoding a xylanase variant was cloned in pET11a in accordance with the procedures for Example 1. Then, using pET11a comprising the DNA encoding a xylanase variant, the expression and purification of the proteins were performed in accordance with the procedures for Example 1 to obtain the xylanase variants (SEQ ID NO: 18, SEQ ID NO: 19, and SEQ ID NO: 20) in Example 3.

TABLE 4
Primer Pairs for Introducing Mutations and Corresponding Xylanase Variants
(Amino Acid Sequence and Base Sequence) Used in Example 3
The underlines indicate nutation sites.

| | Amino Acid | Amino Acid | | | | Endoglucanase Variant | |
|---|---|---|---|---|---|---|---|
| Position | Before Substitution | After Substitution | Fw/Rv | Primer Base Sequence (5′-3′) | | Amino Acid Sequence | Base Sequence |
| Position 78 | Aspartic Acid | Alanine | Fw | TTGCGGTGCGTTTACATCTGGCAAGGGCT | SEQ ID NO: 21 | SEQ ID NO: 18 | SEQ ID NO: 84 |
| | | | Rv | GATGTAAACGCACCGCAATTGACCCAGG | SEQ ID NO: 22 | | |
| Position 35 | Serine | Cysteine | Fw | GATATACATATGTGTATCACGACGAGC | SEQ ID NO: 49 | | |
| | | | Rv | GCTCGTCGTGATACACATATGTATATC | SEQ ID NO: 50 | | |
| Position 44 | Asparagine | Histidine | Fw | ACTGGGACGCACAACGGCTACTACTACTCG | SEQ ID NO: 51 | | |
| | | | Rv | CGAGTAGTAGTAGCCGTTGTGCGTCCCAG | SEQ ID NO: 52 | | |
| Position 62 | Threonine | Cysteine | Fw | GTCACCTACTGTAATGGTGACAATGGCG | SEQ ID NO: 53 | | |
| | | | Rv | TTCGCCATTGTCACCATTACAGTAGGTGAC | SEQ ID NO: 54 | | |
| Position 63 | Asparagine | Leucine | Fw | ACCTACACATTAGGTGACAATGGCGAATAC | SEQ ID NO: 55 | | |
| | | | Rv | GTATTCGCCATTGTCACCTAATGTGTAGG | SEQ ID NO: 56 | | |
| Position 101 | Threonine | Proline | Fw | GGAGAATTTAATCCCAGCGGAAACGCT | SEQ ID NO: 57 | | |
| | | | Rv | AGCGTTTCCGCTGGGATTAAATTCTCC | SEQ ID NO: 58 | | |
| Position 102 | Serine | Asparagine | Fw | GAATTTAATACAAACGGAAACGCTTAT | SEQ ID NO: 59 | | |
| | | | Rv | ATAAGCGTTTCCGTTTGTATTAAATTC | SEQ ID NO: 60 | | |
| Position 61 | Tyrosine | Methion ne | Fw | GAAGTCACCATGACAAATGGTGACAATGGC | SEQ ID NO: 61 | | |
| | | | Rv | GCCATTGTCACCATTTGTCATGGTGACTTC | SEQ ID NO: 62 | | |
| Position 65 | Aspartic Acid | Proline | Fw | CCTACACAAATGGTCCTAACGGCGAATAC | SEQ ID NO: 63 | | |
| | | | Rv | GTATTCGCCGTTAGGACCATTTGTGTAGG | SEQ ID NO: 64 | | |
| Position 66 | Asparagine | Glycine | Fw | ACAAATGGTGATGGAGGCGAATACAGC | SEQ ID NO: 65 | | |
| | | | Rv | GCTGTATTCGCCTCCATCACCATTTGT | SEQ ID NO: 66 | | |
| Position 80 | Threonine | Alanine | Fw | GTGACTTTGCGTCTGGCAAGGGCTGGAATC | SEQ ID NO: 23 | SEQ ID NO: 19 | SEQ ID NO: 85 |
| | | | Rv | TTGCCAGACGCAAAGTCACCGCAATTGAC | SEQ ID NO: 24 | | |
| Position 35 | Serine | Cysteine | Fw | GATATACATATGTGTATCACGACGAGC | SEQ ID NO: 49 | | |
| | | | Rv | GCTCGTCGTGATACACATATGTATATC | SEQ ID NO: 50 | | |
| Position 44 | Asparagine | Histidine | Fw | ACTGGGACGCACAACGGCTACTACTACTCG | SEQ ID NO: 51 | | |
| | | | Rv | CGAGTAGTAGTAGCCGTTGTGCGTCCCAG | SEQ ID NO: 52 | | |
| Position 62 | Threonine | Cysteine | Fw | GTCACCTAGTGTAATGGTGACAATGGCG | SEQ ID NO: 53 | | |
| | | | Rv | TTCGCCATTGTCACCATTACAGTAGGTGAC | SEQ ID NO: 54 | | |
| Position 63 | Asparagine | Leucine | Fw | ACCTACACATTAGGTGACAATGGCGAATAC | SEQ ID NO: 55 | | |
| | | | Rv | GTATTCGCCATTGTCACCTAATGTGTAGG | SEQ ID NO: 56 | | |
| Position 101 | Threonine | Proline | Fw | GGAGAATTTAATCCCAGCGGAAACGCT | SEQ ID NO: 57 | | |
| | | | Rv | AGCGTTTCCGCTGGGATTAAATTCTCC | SEQ ID NO: 58 | | |
| Position 102 | Serine | Asparagine | Fw | GAATTTAATACAAACGGAAACGGTTAT | SEQ ID NO: 59 | | |
| | | | Rv | ATAAGCGTTTCCGTTTGTATTAAATTC | SEQ ID NO: 60 | | |
| Position 61 | Tyrosine | Methionine | Fw | GAAGTCACCATGACAAATGGTGACAATGGC | SEQ ID NO: 61 | | |
| | | | Rv | GCCATTGTCACCATTTGTCATGGTGACTTC | SEQ ID NO: 62 | | |
| Position 65 | Aspartic Acid | Proline | Fw | CCTACACAAATGGTCCTAACGGCGAATAC | SEQ ID NO: 63 | | |
| | | | Rv | GTATTCGCCGTTAGGACCATTTGTGTAGG | SEQ ID NO: 64 | | |
| Position 66 | Asparagine | Glycine | Fw | ACAAATGGTGATGGAGGCGAATACAGC | SEQ ID NO: 65 | | |
| | | | Rv | GCTGTATTCGCCTCCATCACCATTTGT | SEQ ID NO: 66 | | |
| Position 78 | Aspartic Acid | Alanine | Fw | TTGCGGTGCGTTTACATCTGGGAAGGGCT | SEQ ID NO: 21 | SEQ ID NO: 20 | SEQ ID NO: 86 |
| | | | Rv | GATGTAAACGCACCGCAATTGACCCAGG | SEQ ID NO: 22 | | |
| Position 155 | Arginme | Alanine | Fw | CAACACAGGCGGTCGACCAACCCTCCATC | SEQ ID NO: 27 | | |
| | | | Rv | GGTCGACCGCCTGTGTTGAGTAGATATCG | SEQ ID NO: 28 | | |
| Position 35 | Serine | Cysteine | Fw | GATATACATATGTGTATCACGACGAGC | SEQ ID NO: 49 | | |
| | | | Rv | GCTCGTCGTGATACACATATGTATATC | SEQ ID NO: 50 | | |
| Position 44 | Asparagine | Histidine | Fw | ACTGGGACGCACAACGGCTACTACTACTCG | SEQ ID NO: 51 | | |
| | | | Rv | CGAGTAGTAGTAGCCGTTGTGCGTCCCAG | SEQ ID NO: 52 | | |
| Position 62 | Threcnine | Cysteine | Fw | GTCACCTACTGTAATGGTGACAATGGCG | SEQ ID NO: 53 | | |
| | | | Rv | TTCGCCATTGTCACCATTACAGTAGGTGAC | SEQ ID NO: 54 | | |
| Position 63 | Asparagine | Leucine | Fw | ACCTACACATTAGGTGACAATGGCGAATAC | SEQ ID NO: 55 | | |
| | | | Rv | GTATTCGCCATTGTCACCTAATGTGTAGG | SEQ ID NO: 56 | | |
| Position 101 | Threcnine | Proline | Fw | GGAGAATTTAATCCCAGCGGAAACGCT | SEQ ID NO: 57 | | |
| | | | Rv | AGCGTTTCCGCTGGGATTAAATTCTCC | SEQ ID NO: 58 | | |
| Position 102 | Serine | Asparagine | Fw | GAATTTAATACAAACGGAAACGCTTAT | SEQ ID NO: 59 | | |
| | | | Rv | ATAAGCGTTTCCGTTTGTATTAAATTC | SEQ ID NO: 60 | | |
| Position 61 | Tyrosine | Methionine | Fw | GAAGTCACCATGACAAATGGTGACAATGGC | SEQ ID NO: 61 | | |
| | | | Rv | GCCATTGTCACCATTTGTCATGGTGACTTC | SEQ ID NO: 62 | | |
| Position 65 | Aspartic Acid | Proline | Fw | CCTACACAAATGGTCCTAACGGCGAATAC | SEQ ID NO: 63 | | |
| | | | Rv | GTATTCGCCGTTAGGACCATTTGTGTAGG | SEQ ID NO: 64 | | |
| Position 66 | Asparagine | Glycine | Fw | ACAAATGGTGATGGAGGCGAATACAGC | SEQ ID NO: 65 | | |
| | | | Rv | GCTGTATTCGCCTCCATCACCATTTGT | SEQ ID NO: 66 | | |

### Example 4: Xylanase Variants of Examples 1 to 3 and Wild-Type Xylanase of Comparative Example 1, and Sugar Solution Production 1 with Xylanase Variant of Comparative Example 2

An attempt was made to produce an oligosaccharide solution using Birchwood xylan (Sigma-Aldrich) as a substrate. The xylanases used were various xylanase variants (Example 1, Example 2, and Example 3), a wild-type xylanase (Comparative Example 1), and a xylanase variant (Comparative Example 2). The substrate, 0.5 g each, was weighed out into a 2 mL tube, water was added so that the final concentration of the biomass became 5% (w/w), and then, the resulting solution was adjusted to pH 5 using diluted hydrochloric acid. After the pH adjustment, the wild-type xylanase or the xylanase variant in an amount of 0.8 mg per g of dry biomass was added to the composition, and the resulting mixture was allowed to react using a thermoblock rotator (SN-48BN manufactured by Nissin Rika Co., Ltd.) for 24 hours. The protein concentration was measured and adjusted in accordance with Reference Example 1. Reactions were carried out for the xylanase variant of Example 3 at 50° C., and for the other xylanase variants at 40° C. The sugar composition of the supernatant obtained after the reaction was analyzed in accordance with Reference Example 3 and put together in Table 5.

As a result, the xylanase variant comprising substitutions at positions selected from position 78, position 80, position 117, position 155, position 169, and position 203 showed an increased concentration of xylotriose and a decreased concentration of xylose compared with the wild-type xylanase. This revealed that the mutations suppressed the decomposition of xylotriose. In particular, the xylanase variant comprising substitutions at position 78, and position 155 and also comprising substitutions at position 35, position 44, position 62, position 63, position 101, position 102, position 61, position 65, and position 66 showed a high concentration of xylotriose and a low concentration of xylose. In contrast, the xylanase variants comprising substitutions at positions selected from position 48, position 112, position 121, position 123, position 128, position 167, position 171, and position 212 did not produce xylotriose or xylose.

TABLE 5
Sugar Solution Production 1 with Xylanase

| | Xylotriose | Xylose |
|---|---|---|
| Wild-type (Comparative Example 1) | 2.15 | 1.82 |
| D78A (Example 1) | 7.17 | 0.41 |
| T80A (Example 1) | 3.06 | 0.94 |
| L117N (Example 1) | 3.01 | 1.12 |
| R155A (Example 1) | 3.15 | 0.92 |
| Q169A (Example 1) | 3.29 | 0.72 |
| N203W (Example 1) | 2.32 | 1.33 |
| D78A/R155A (Comparative Example 2) | 3.06 | 1.39 |
| Y48A (Comparative Example 2) | — | — |
| W112A (Comparative Example 2) | — | — |
| Y121A (Comparative Example 2) | — | — |
| L123Y (Comparative Example 2) | — | — |
| T128Y (Comparative Example 2) | — | — |
| F167A (Comparative Example 2) | — | — |
| W171A (Comparative Example 2) | — | — |
| Y212A (Comparative Example 2) | — | — |
| S35C/N44H/Y61M/T62C/ | 7.06 | 1.05 |
| N63L/T101P/S102N/D65P/ | | |
| N66G/D78A (Example 3) | | |
| S35C/N44H/Y61M/T62C/ | 3.99 | 1.54 |
| N63L/T101P/S102N/D65P/ | | |
| N66G/T80A (Example 3) | | |
| S35C/N44H/Y61M/T62C/ | 7.79 | 0.65 |
| N63L/T101P/S102N/D65P/ | | |
| N66G/D78A/R155A (Example 3) | | |
| (g/L) | | |

### Example 5: Method 2 of Producing Sugar Solution Using Xylanase Variant

An attempt was made to produce an oligosaccharide solution using bagasse, which is a residue obtained by squeezing sugarcane, as a raw material. The xylanase used was the wild-type xylanase of Comparative Example 1 or the xylanase variant of Example 3 having the amino acid sequence of SEQ ID NO: 20. In pre-treatment, dipping treatment was performed in a 1 N caustic soda aqueous solution for six days so that the biomass weight became 30% (w/w). The pretreated material, 0.05 g each, was weighed out into a 2 mL tube, water was added so that the final concentration of the biomass became 5% (w/w), and then the resulting solution was adjusted to pH 5 using diluted hydrochloric acid. After the pH adjustment, the wild-type xylanase or the xylanase variant in an amount of 0.8 mg per g of dry biomass was added to the composition, and the resulting mixture was allowed to react using a thermoblock rotator (SN-48BN manufactured by Nissin Rika Co., Ltd.) at 50° C. for 24 hours. The sugar composition of the supernatant obtained after the reaction was analyzed in accordance with Reference Example 3 and shown in Table 6. Xylotriose, which is xylooligosaccharide, could be obtained in larger amount using the xylanase variant compared with the wild-type xylanase.

TABLE 6
Sugar Solution Production 2 with Xylanase Variant

| | Xylotriose | Xylose |
|---|---|---|
| Wild-type (Comparative Example 1) | 0.23 g/L | 1.27 g/L |
| S35C/N44H/Y61M/T62C/ | 2.52 g/L | 0.6 g/L |
| N63L/T101P/S102N/D65P/ | | |
| N66G/D78A/R155A Variant | | |
| (Example 3) | | |

### Example 6: Residual Activity of Xylanase After Reaction in Example 5

The supernatant in an amount of 50 μl after the reaction obtained in Example 5 was ultrafiltrated using the VIVASPIN500 (PES, a molecular weight cutoff of 10,000) (Sartorius) to thereby collect the wild-type xylanase of Comparative Example 1 or the xylanase variant of Example 3. The xylanase activities of the following were measured: the collected wild-type xylanase of Comparative Example 1 or the collected xylanase variant of Example 3 having the amino acid sequence of SEQ ID NO: 20; and the wild-type xylanase of Comparative Example 1 or xylanase variant of Example 3 having the amino acid sequence of SEQ ID NO: 20, diluted to the concentration for xylan decomposition (40 mg/1). The xylanase activity was measured using 1% Birchwood xylan (Sigma-Aldrich Co. LLC) as a substrate. The xylanase hydrolyzed the Birchwood xylan, and the amount of the produced reducing sugar was measured by the dinitrosalicylic acid (DNS) method using xylose as a standard sample. The collected xylanase or diluted xylanase was added in an amount of 1/10 of the reaction solution, and decomposition of the Birchwood xylan was performed by maintaining the solution at a temperature of 50° C. for ten minutes. After the reaction, for the measurement of the reducing sugar amount, 0.75 mL of DNS solution was added to start a reaction, and the reaction solution was boiled for five minutes to terminate the reaction. The reaction solution obtained after termination of the reaction was subjected to measurement of absorbance at 540 nm to measure the reducing sugar amount. One unit of xylanase activity was defined as the amount of enzyme needed to produce 1 μmol of xylose from Birchwood xylan at 50° C. in one minute, and the number of units was calculated. The relative activity values of the collected wild-type xylanase of Comparative Example 1 and xylanase variant of Example 3 were determined as the residual activity using the activity values of the diluted wild-type xylanase of Comparative Example 1 and xylanase variant of Example 3, respectively, as a standard (100%). The residual activity after the collection are shown in Table 7. The activity of the xylanase variant was high even after decomposition of the xylan, and it was possible to verify that the xylanase variant can be reused effectively for decomposing xylan.

TABLE 7
Residual Activity After Bagasse Decomposition with
Xylanase Variant

| | Residual Activity |
|---|---|
| Wild-type (Comparative Example 1) | 5% |
| S35C/N44H/Y61M/T62C/ | 70% |
| N63L/T101P/S102N/D65P/ | |
| N66G/D78A/R155A Variant | |
| (Example 3) | |

### Comparative Example 3: Production of Sugar Solution with Wild-type Xylanase (Having Amino Acid Sequence of SEQ ID NO: 88) Derived from Trichoderma Reesei

A DNA fragment (SEQ ID NO: 92) having the base sequence of a protein obtained by removing the signal peptide region from the wild-type xylanase derived from Trichoderma reesei was synthesized and cloned in pET11a in accordance with the procedures for Example 1. Then, using pET11a comprising the DNA encoding a xylanase variant, the protein was expressed and purified in accordance with the procedures for Comparative Example 1 to obtain the wild-type xylanase (SEQ ID NO: 88). Furthermore, the sugar solution was produced in accordance with the procedures for Example 4, and the sugar composition of the supernatant after the reaction is shown in Table 8.

### Example 7: Production of Sugar Solutions with Xylanase Variants (Having Amino Acid Sequences of SEQ ID NOs: 89 and 90) Derived from Trichoderma Reesei

An alignment of amino acids was carried out between the wild-type xylanase (SEQ ID NO: 87) derived from Trichoderma reesei and SEQ ID NO: 1, and the positions in SEQ ID NO: 87 corresponding to positions 78 and 80 in SEQ ID NO: 1 were determined. The alignment was carried out in accordance with Procedures 1) to 3) described above using the Parallel Editor (Multiple Allignment) in Genetyx, a program included in ClustalW, with the default parameters. A DNA fragment having the base sequence of SEQ ID NO: 93 encoding a xylanase having a substitution from aspartic acid to alanine at position 78, and a DNA fragment having the base sequence of SEQ ID NO: 94 encoding a xylanase having a substitution from valine to alanine at position 80, in a protein obtained by removing the signal peptide region from a xylanase variant derived from Trichoderma reesei were synthesized. Cloning, protein expression, and purification were carried out in the same manner as in Comparative Example 3 to obtain xylanase variants (SEQ ID NOs: 89 and 90). The relationship between substitution sites and substitution residues is shown in Table 9. Furthermore, the sugar solution was produced in accordance with the procedures for Example 4, and the sugar composition of the supernatant after the reaction is shown in Table 8. Xylotriose, which is xylooligosaccharide, could be obtained in larger amount using the xylanase variant compared with the wild-type xylanase.

TABLE 8

| | Amino Acid Residue at | Amino Acid Residue at | | |
|---|---|---|---|---|
| | Position in SEQ ID NO: 87 | Position in SEQ ID NO: 87 | SEQ ID NO: | SEQ ID NO: |
| | Corresponding to Position 78 in | Corresponding to Position 80 in | (Amino Acid | (Base |
| | SEQ ID NO: 1 | SEQ ID NO: 1 | Sequence) | Sequence) |
| Wild-type | Aspartic Acid | Valine | SEQ ID NO: 88 | SEQ ID NO: 92 |
| Variant 1 | Alanine | Valine | SEQ ID NO: 89 | SEQ ID NO: 93 |
| Variant 2 | Aspartic Acid | Alanine | SEQ ID NO: 90 | SEQ ID NO: 94 |

TABLE 9
Sugar Solution Production with
Xylanase Derived from Trichoderma reesei

| | Xylotriose | Xylose |
|---|---|---|
| Wild-type | 2.5 | 2.0 |
| Variant 1 | 4.7 | 0.9 |
| Variant 2 | 3.2 | 1.3 |

(g/L)

### INDUSTRIAL APPLICABILITY

Since the decomposition of xylotriose is suppressed, the xylanase variant can produce oligosaccharide from xylan at a high yield, and accordingly, can be used for hydrolysis of biomass, production of sugar solution, and production of oligosaccharide.

All publications, patents, and patent applications cited herein are incorporated herein by reference in their entirety.

1-14. (canceled)

15. A xylanase variant derived from a filamentous fungus, comprising an amino acid sequence,
wherein one or more amino acid residues at the positions selected from the positions corresponding to position 78, position 117, and position 203 in the amino acid sequence of SEQ ID NO: 1 are each substituted with a different amino acid residue,
the amino acid residue at the position corresponding to position 78 is substituted with alanine, glycine, valine, leucine, or isoleucine, and
said xylanase variant having a xylanase activity whereby the productivity of xylotriose from xylan is improved compared with the original xylanase wherein said one or more amino acid residues are not substituted.

16. The xylanase variant according to claim 28, comprising an amino acid sequence wherein the amino acid residue at a position corresponding to position 80 in the amino acid sequence of SEQ ID NO: 1 is substituted with alanine, glycine, leucine, or isoleucine.

17. The xylanase variant according to claim 15, comprising an amino acid sequence wherein the amino acid residue at a position corresponding to position 117 in the amino acid sequence of SEQ ID NO: 1 is substituted with serine, threonine, or asparagine.

18. The xylanase variant according to claim 28, comprising an amino acid sequence wherein the amino acid residue at a position corresponding to position 155 in the amino acid sequence of SEQ ID NO: 1 is substituted with alanine, glycine, valine, leucine, or isoleucine.

19. The xylanase variant according to claim 28, comprising an amino acid sequence wherein the amino acid residue at a position corresponding to position 169 in the amino acid sequence of SEQ ID NO: 1 is substituted with alanine, glycine, valine, leucine, or isoleucine.

20. The xylanase variant according to claim 15, comprising an amino acid sequence wherein the amino acid residue at a position corresponding to position 203 in the amino acid sequence of SEQ ID NO: 1 is substituted with tryptophan, phenylalanine, or tyrosine.

21. The xylanase variant according to claim 28, comprising an amino acid sequence wherein the amino acid residue at a position corresponding to position 78 in the amino acid sequence of SEQ ID NO: 1 is substituted with alanine, and the amino acid residue at a position corresponding to position 155 in the amino acid sequence of SEQ ID NO: 1 is substituted with alanine.

22. The xylanase variant according to claim 15, wherein said original xylanase is a filamentous fungus-derived xylanase belonging to Glycoside Hydrolase Family 11.

23. The xylanase variant according to claim 15, comprising any one of the amino acid sequences of (a) to (c), and having a xylanase activity:
(a) any one of the amino acid sequences shown in SEQ ID NOs: 3, 5, 8, 9, 89, and 90;
(b) an amino acid sequence derived from any one of the amino acid sequences shown in SEQ ID NOs: 3, 5, 8, 9, 89, and 90, wherein the amino acids substituted at the positions corresponding to position 78, position 117, position 155, and position 203 in the amino acid sequence of SEQ ID NO: 1 are not mutated, and one to several amino acids are deleted, substituted, inserted, or added at positions of amino acids other than said substituted amino acids; or
(c) an amino acid sequence derived from any one of the amino acid sequences shown in SEQ ID NOs: 3, 5, 8, 9, 89, and 90, wherein the amino acids substituted at the positions corresponding to position 78, position 117, position 155, and position 203 in the amino acid sequence of SEQ ID NO:1 are not mutated, and said amino acid sequence except said substituted amino acids has an amino acid identity of 90% or more to said any one of the amino acid sequences.

24. The xylanase variant according to claim 15, wherein the amino acid residues at positions corresponding to position 35, position 44, position 61, position 62, position 63, position 65, position 66, position 101, and position 102 in the amino acid sequence of SEQ ID NO: 1 are further each substituted with a different amino acid residue.

25. The xylanase variant according to claim 24, wherein:
the amino acid residue at a position corresponding to position 35 in SEQ ID NO: 1 is substituted with cysteine,
the amino acid residue at a position corresponding to position 44 in SEQ ID NO: 1 is substituted with histidine,
the amino acid residue at a position corresponding to position 61 in SEQ ID NO: 1 is substituted with methionine,
the amino acid residue at a position corresponding to position 62 in SEQ ID NO: 1 is substituted with cysteine,
the amino acid residue at a position corresponding to position 63 in SEQ ID NO: 1 is substituted with leucine,
the amino acid residue at a position corresponding to position 65 in SEQ ID NO: 1 is substituted with proline,
the amino acid residue at a position corresponding to position 66 in SEQ ID NO: 1 is substituted with glycine,
the amino acid residue at a position corresponding to position 101 in SEQ ID NO: 1 is substituted with proline, and
the amino acid residue at a position corresponding to position 102 in SEQ ID NO: 1 is substituted with asparagine.

26. The xylanase variant according to claim 24, comprising any one of the amino acid sequences of (d) to (f), and having a xylanase activity:
(d) any one of the amino acid sequences shown in SEQ ID NOs: 18 to 20;
(e) an amino acid sequence derived from any one of the amino acid sequences shown in SEQ ID NOs: 18 to 20, wherein the amino acids substituted at the positions corresponding to position 35, position 44, position 61, position 62, position 63, position 65, position 66, position 78, position 80, position 101, position 102, and position 155 in the amino acid sequence of SEQ ID NO: 1 are not mutated, and one to several amino acids are deleted, substituted, inserted, or added at positions of amino acids other than said substituted amino acids; or
(f) an amino acid sequence derived from any one of the amino acid sequences shown in SEQ ID NOs: 18 to 20, wherein the amino acids substituted at the positions corresponding to position 35, position 44, position 61, position 62, position 63, position 65, position 66, position 78, position 80, position 101, position 102, and position 155 in the amino acid sequence of SEQ ID NO: 1 are not mutated, and said amino acid sequence except said substituted amino acids has an amino acid identity of 90% or more to said any one of the amino acid sequences.

27. An enzyme composition comprising the xylanase variant according to claim 15.

28. The xylanase variant according to claim 15, wherein one or more amino acid residues at positions corresponding to position 80, position 155, and position 169 in the amino acid sequence of SEQ ID NO: 1 is further substituted.
